(19) United States
(12) Patent Application Publication
(10) Pub. No.: US 2005/0261472 A1
(43) Pub. Date: Nov. 24, 2005

(54) Binding moieties for fibrin

(75) Inventors: Charles R Wescott, Belmont, MA (US); James P. Beltzer, Carlisle, MA (US); Shrikumar A. Nair, Lexington, MA (US); Andrew Kolodziej, Winchester, MA (US)

Correspondence Address: FISH & RICHARDSON PC, P.O. BOX 1022, MINNEAPOLIS, MN, 55440-1022, US

(73) Assignees: Dyax Corp.; Epix Medical, Inc., Massachusetts corporations

(21) Appl. No.: 10/649,229

(22) Filed: Aug. 26, 2003

Related U.S. Application Data
(63) Continuation of application No. 09/627,806, filed on Jul. 28, 2000, now abandoned.
(60) Provisional application No. 60/146,425, filed on Jul. 29, 1999.

(51) Int. Cl. C07K 7/08; C07K 7/06

(52) U.S. Cl. 530/328; 530/329

(57) ABSTRACT

The present invention provides binding moieties for fibrin, which have a variety of uses wherever detecting, isolating or localizing fibrin, and particularly fibrin as opposed to fibrinogen, is advantageous. Particularly disclosed are synthetic, isolated polypeptides capable of binding fibrin and recognizing the form of polymerized fibrin found in thrombi. Such polypeptides and disclosed derivatives are useful, e.g., as imaging agents for thrombi. Preferred embodiments useful as magnetic resonance imaging (MRI) contrast agents useful for detecting a thrombus in vivo are also disclosed.

### CROSS-REFERENCE TO RELATED APPLICATION

This application claims priority to U.S. provisional application No. 60/146,425, filed Jul. 29, 2000.

### FIELD OF THE INVENTION

The present invention relates to fibrin-binding polypeptides and compositions for detection and treatment of pathological intravascular thrombosis. More particularly, the invention relates to materials useful for and methods of detecting, imaging, and localizing thrombi. The invention provides binding moieties capable of distinguishing between fibrin and circulating fibrinogen and defining a unique epitope on polymerized fibrin. Such binding moieties are useful for the detection, imaging and localization of fibrin-containing clots by magnetic resonance imaging and are also useful in the diagnosis and treatment of coronary conditions where fibrin plays a role. Screening methods for the isolation of fibrin binding moieties are also disclosed.

### BACKGROUND OF THE INVENTION

Thrombus associated diseases are vascular conditions that are developed due to the presence of a clot. Such diseases are a major cause of mortality in patients, and therefore developing thrombus-specific diagnosis, treatment, and detection methodologies and reagents is of great clinical importance. Pulmonary embolism (PE), deep-vein thrombosis, stroke, and atherosclerosis are examples of thrombus associated diseases.

Deep-vein thrombosis is a condition in which blood clots form in the deep blood vessels of the legs and groin. These clots can block the flow of blood from the legs back to the heart. Sometimes, a piece of a clot is detached and carried by the bloodstream through the heart to a blood vessel, where it lodges and reduces, or blocks, the flow of blood to a vascular tissue. This is called an embolism. Such a clot lodging in a blood vessel in the lung is a pulmonary embolism, or PE. PE can cause shortness of breath, chest pain, or even death.

In the United States alone, there are an estimated 600,000 patients that suffer pulmonary embolism each year. In approximately 378,000 of these patients, PE goes undetected, and approximately 114,000 of these patients die later due to complications associated with the disease. This high mortality is partly due to the absence of clinical symptoms in many cases and to the significant limitations associated with currently available methods of investigation and detection.

There is a need, therefore, for sensitive and effective assays to detect the presence of venous thromboembolism at various stages of development, for ways to diagnose the presence or absence of early and late thrombi, and for non-invasive reagents that can specifically bind thrombi and which will be useful for detecting the presence or absence of the early or late thrombus in patients.

Thrombus Formation

Crosslinked fibrin forms the underlying backbone of both venous and arterial clots or thrombi (Harker et al., Am. J. Cardiology, 60:20B-28B (1987)). Thrombi are formed when the enzyme thrombin is activated, leading to cleavage of plasma fibrinogen to release fibrinopeptides and expose a fibrin polymerization site (Hermans et al., Semin. Thromb. Hemost., 8:11-24 (1982)).

The biology of fibrin and clot formation has been investigated by many researchers in recent years, and a detailed understanding of the cascade of events leading to clot formation has emerged. There are two major activation pathways for coagulation: the intrinsic pathway which requires Factors XII, IX and VIII and the extrinsic pathway which involves tissue factor and Factor VIII. Both pathways converge at the point of activating Factor X, the enzyme responsible for converting prothrombin to thrombin.

The extrinsic pathway is initiated by tissue factor, a ubiquitous cellular lipoprotein which forms a calcium-dependent complex with Factor VII. Upon complex formation, Factor VII is activated to Factor VIIa, which converts Factor X to Factor Xa. Factor Xa converts prothrombin to thrombin in conjunction with Factor Va, calcium and phospholipid. Prothrombin conversion also occurs on endothelial surfaces and activated platelets, and requires the assembly of a complex between Factor Xa, Factor Va, and prothrombin. This conversion requires the presence of phospholipid and calcium ions.

The intrinsic or contact coagulation pathway is initiated by platelets. The cascade begins with the formation of a complex among Factor XII, high molecular weight kininogen, and prekallikrein. Upon complex formation, Factor XII is cleaved to Factor XIIa. After the stepwise activation of Factors XI, IX, VIII, X, and V, as in the extrinsic pathway, prothrombin is activated to thrombin. Thrombin, which is a trypsin-like serine protease, is the central regulator of hemostasis and thrombosis. Fibrin is derived from fibrinogen, and polymerization of fibrin occurs following enzymatic cleavage of fibrinogen by thrombin. Fibrinogen (340 kD) consists of three pairs of identical peptides, designated Aα, Bβ, and γ. Chemical structural analysis and electron microscopy have demonstrated that the protein has a trinodular structure. Two AαBβγ subunits are oriented in an antiparallel configuration. The amino terminal portions of the six chains are bundled together in a central “E” domain. Two coiled-coil strands extend outward from either side of the E domain to the two terminal nodes, the “D” domains. These coiled coil regions are 110 amino acids long and composed of all three chains. The D domains contain two high affinity Ca²⁺ binding sites and are involved with the E domain in fibrin polymerization. Extensive disulfide bridges covalently crosslink the two subunits, and stabilize the globular domains. The C-terminal portions of the Aa chains form flexible extensions beyond the D domains. The D domain contains Factor XIIIa crosslinking sites and is the primary site of plasmic digestion during fibrinolysis.

Fibrin formation from fibrinogen is a spontaneous self-assembly process resulting from the removal of fibrinopeptides by thrombin. Thrombin cleavage at the Arg16-Arg17 bond in the Ax chains and at the Arg14-Gly15 bond on the BP chains releases fibrinopeptides A and B, and exposes a polymerization site in the E domain consisting mainly of the N-terminus of the α chain. This N-terminus, which bears the sequence Gly-Pro-Arg-Val, binds to a complementary polymerization site on two adjacent fibrinogen chains. End to end association of these fibrinogen molecules mediated by the D domains, creates a binding site for the E domain polymerization site, located on a third fibrinogen molecule. This DD(E) ternary complex forms a core that stabilizes the forming fibrin gel. The initial polymerization product is a linear, two-stranded protofibril. Lateral coalescence of these protofibrils results in thick fibers and a branched, three dimensional matrix. Lateral assembly is complex but probably involves the B polymerization site (the N-terminus of β) and trimolecular complexes formed through D domain interactions.

Adjacent fibrin monomers within the fibrils become covalently crosslinked by Factor XIIIa, a plasma transglutaminase which is itself activated by thrombin and fibrin. These crosslinks add mechanical stability to the fibrin network and increase resistance to clot degradation. Factor XIIIa also enhances clot stability by crosslinking specialized proteins to fibrin, including the plasmin inhibitor α₂ antiplasmin, and the adhesion protein fibronectin.

Thrombus Imaging

The search for thrombus-specific imaging agents began two decades ago when radiolabeled fibrinogen was first evaluated (Kakkar et al., Lancet, 1:540-542 (1970)). Since then a number of thrombus imaging agents have been described, including agents that are incorporated into forming thrombi and agents that bind to components of previously formed thrombi (Knight et al., Radiology, 156:509-514 (1985); Alavi et al., Radiology, 175:79-85 (1990); Rosebrough et al., J. Nuc. Med. 31:1048-1054 (1990)). Among the recent approaches that have been taken in the development of materials useful for visualizing or imaging thrombi are radiolabeled platelets and anti-platelet antibodies that bind to forming thrombi, anti-fibrin antibodies, anti-activated platelet antibodies, and activated or inactivated tissue type plasminogen activator (tPA) (Thakur et al., Throm. Res., 9:345-357 (1976); Palabrica et al., Proc. Natl. Acad. Sci., 86:1036-1040 (1989)).

Platelet affinity peptides have also been used to detect clots. This approach utilizes small peptides capable of binding to platelets which are labeled with ^{99m}Tc. The platelets, with labeled peptide attached, become incorporated into thrombi and render the thrombi detectable (Knight et al., J. Nucl. Med., 35:195-202 (1994); Muto et al., Radiology, 189 (suppl):303(1993)).

Because platelets in thrombi degrade over time, the use of platelet affinity peptides, anti-platelet antibodies and other agents that bind to platelets or that detect platelet location are only useful for detection of early clots (less than 12 hours) and cannot be used in detection and imaging of embolism, particularly pulmonary embolism.

Since Fibrin is the major protein component in thrombi it is thus a desirable target for agents that can mark the location and gauge the size of a clot in a subject. Fibrin targeting, however, is complicated by the close structural similarity between fibrin and its circulating precursor, fibrinogen. One successful approach has involved the isolation of monoclonal antibodies specific to fibrin. One such class of monoclonals recognizes the newly exposed N-termini of the α and β chains of the fibrin monomers. Another class of monoclonal antibodies recognizes epitopes exposed as a result of polymerization, such as the covalent crosslinks formed by Factor XIII, the DD dimer domain, or the putative tPA binding site. The use of antibodies as imaging agents does, however, have some disadvantages: The high molecular weight of antibodies necessitates that a larger mass of agent must be delivered to a clot than would be required of a small molecule, and this may be a serious-limitation when higher concentrations of an imaging agent are essential to obtaining adequate signal contrast. Labeled antibodies often present clearance problems because of relatively long circulating half-lives in vivo, limiting contrast with the blood and tissue background. In addition, antibodies are often expensive to prepare and formulate, and their use can lead to undesirable and potentially fatal immunogenic responses.

Another method used for pulmonary embolism diagnosis is the ventilation/perfusion scan. In a ventilation/perfusion scan, the patient inhales a radiographic gas, and images of regions of the lung that are capable of ventilation are recorded. Subsequently, the patient is injected with a radioactive agent and the movement of the agent through the pulmonary artery is traced. The two images are compared, and any area of thrombosis is detected by contrasting the ventilation data with the perfusion data. Approximately 930,000 ventilation/perfusion scans are performed every year in the United States, but approximately 60% are inconclusive.

An alternate method for pulmonary embolism diagnosis is x-ray angiography. This method is performed by introducing an x-ray opaque (radiopaque) compound proximally to the heart or pulmonary artery via arterial catheter introduced through the patient's femoral artery. The compound is traced through the pulmonary artery by an x-ray camera and thrombosis is detected by such tracing. Although this method is considered a “gold standard” test by clinicians and approximately 60,000 angiographies are performed annually in the United States, the test is invasive and expensive. Moreover, 1 out of 200 patients undergoing the x-ray angiography dies as a direct result of the procedure itself.

Magnetic resonance imaging (MRI) has previously been used for diagnosis purposes. However, such methods currently only detect thrombi as ambiguous negative signal areas, which can be misdiagnosed as anatomical irregularities. Although new image analysis software is improving the thrombus-imaging capability of MRI, MRI contrast agents are currently unavailable that can localize a thrombus by rendering a positive image. Thus, a need exists for a MRI contrast agent that provides a positive image of a thrombus.

### SUMMARY OF THE INVENTION

In answer to the need for improved materials and methods for detecting, localizing, measuring and treating fibrin clots, we have now surprisingly discovered a group of non-naturally occurring polypeptides that bind specifically to fibrin. Appropriate labeling of such polypeptides provides detectable imaging agents that bind at high concentration to a clot, providing excellent thrombus specific imaging agents. Conjugation or fusion of such polypeptides with effective agents such as thrombolytics can be used to treat thrombotic conditions, e.g., by causing the conjugate or fusion to “home” to the site of a fibrin clot, thereby providing an effective treatment for thrombus associated diseases. Recombinant bacteriophage displaying the fibrin-binding polypeptides of the invention have been identified and isolated, and such phage products are also valuable reagents for effective detection and diagnosis of thrombi.

In addition to the detection of thromboembolism and thrombus formation, e.g., on atherosclerotic plaque, the newly discovered fibrin binders can also be used advantageously to detect numerous other pathophysiologies in which fibrin plays a role. In these cases, fibrin imaging can be a useful direct or surrogate marker for diagnosis or therapeutic monitoring. For example, peritoneal adhesions often occur after surgery or inflammatory and neoplastic processes, and are comprised of a fibrin network, fibroblasts, macrophages, and new blood vessels. Patients suffering from rheumatoid arthritis, lupus, or septic arthritis often have bits of fibrin-containing tissues called rice bodies in the synovial fluid of their joints. In thrombotic thrombocytopenic purpura, a type of anemia, fibrin deposits in arterioles cause turbulent blood flow, resulting in stress and destruction of the red blood cells. The fibrin binding moieties of the instant invention can be used in the detection and diagnosis of such fibrin-related disorders.

The fibrin specific agents can also be used to detect other conditions including but not limited to hypoxia or ischemia of the heart, kidney, liver, lung, brain, or other organs, as well as the detection of tumors, diabetic retinopathy, early or high-risk atherosclerosis, and other autoimmune and inflammatory disorders. Fibrin specific agents also could provide both direct or surrogate markers of disease models in which hypoxia and angiogenesis are expected to play a role. In hypoxic conditions, fibrin(ogen) is expressed under the control of hypoxia-inducible factor 1 (HIF-1). In those disease models where angiogenesis plays a role, such as tumor growth and invasion, fibrin provides the structural mesh required for the laying down of new blood vessels.

This invention pertains to fibrin binding moieties. Binding moieties according to this invention are useful in any application where binding, detecting or isolating fibrin or its fragments (e.g., DD and DD (E)) is advantageous. A particularly advantageous use of the binding moieties disclosed herein is in a method of imaging thrombi in vivo. The method entails the use of fibrin specific binding moieties according to the invention for detecting a thrombus, where the binding moieties have been detectably labeled for use as imaging agents, including magnetic resonance imaging (MRI) contrast agents, x-ray imaging agents, radiopharmaceutical imaging agents, ultrasound imaging agents, and optical-imaging agents.

The most preferred fibrin binding moieties according to the invention are isolated, synthetic polypeptides having a high affinity for fibrin. This invention provides a new class of fibrin binding polypeptides having an amino acid sequence comprising:
X₁—X₂-Cys-X₄—X₅-Tyr-X₇—X₈Cys-X₁₀—X₁₁ (SEQ ID NO:1), wherein
X₁ is Arg, Asp, His, Leu, or Phe;
X₂ is Ala, Asp, Gly, Pro, or Ser;
X₄ is Ala, Glu, Phe, Gly, Ile, Lys, Leu, Met, Arg, Thr, Val, Tyr, Asn, Asp, Gln, His, Ser, or Trp;
X₅ is Ala, Tyr, Phe, or Ser;
X₇ is Gly, Ala, or DAla;
X₈ is Thr, Val, or Ser;
X₁₀ is His, Leu, or Phe;
X₁₁ is Arg, Asp, or His,
   wherein said polypeptide has the ability to bind fibrin.

In particular, a stable binding loop having a high affinity for fibrin is disclosed, having the formula: Cys-X₂—X₃-Tyr-X₅—X₆-Cys (SEQ ID NO: 2), wherein
X₂ is Ala, Glu, Phe, Gly, Ile, Lys, Leu, Met, Arg, Thr, Val, Tyr, Asn, Asp, Gln, His, Ser, or Trp;
X₃ is Ser, Phe, Ala, or Tyr;
X₅ is Gly, Ala, or DAla; and
X₆ is Thr, Val, or Ser.

Preferred polypeptides according to the invention comprise an amino acid sequence:
Cys-Xaa-Tyr-Tyr-Gly-Thr-Cys (SEQ ID NO:3), where Xaa is Asn, Asp, Gln, His, Ser or Trp,
Tyr-Tyr-Gly-Xaa (SEQ ID NO: 64), where Xaa is Thr, Ser or Val,
Tyr-Tyr-Gly-Thr (SEQ ID NO:4),
Arg-Ser-Cys-Asn-Tyr-Tyr-Gly-Thr-Cys-Leu-His (SEQ ID NO:5);
His-Asp-Cys-Gln-Tyr-Tyr-Gly-Thr-Cys-Leu-His (SEQ ID NO:6);
Phe-Ala-Cys-His-Tyr-Tyr-Gly-Thr-Cys-Leu-His (SEQ ID NO:7);
Arg-Pro-Cys-Asp-Tyr-Tyr-Gly-Thr-Cys-Phe-Asp (SEQ ID NO:8);
Leu-Pro-Cys-Asp-Tyr-Tyr-Gly-Thr-Cys-Leu-Asp (SEQ ID NO:9);
Phe-Ser-Cys-Trp-Tyr-Ser-Leu-His-Cys-His-Arg (SEQ ID NO:10);
Asp-Pro-Cys-Ser-Tyr-Tyr-Gly-Thr-Cys-Leu-His (SEQ ID NO:11);
Leu-Pro-Cys-Ser-Tyr-Tyr-Gly-Thr-Cys-Leu-His (SEQ ID NO:12);
Leu-Ser-Cys-Asp-Tyr-Tyr-Gly-Thr-Cys-Leu-Arg (SEQ ID NO:13);
Leu-Ala-Cys-His-Tyr-Tyr-Gly-Thr-Cys-Leu-His (SEQ ID NO:14);
Asp-Gly-Cys-His-Tyr-Tyr-Gly-Thr-Cys-Leu-His (SEQ ID NO:15);
Arg-Pro-Cys-Asn-Tyr-Tyr-Gly-Thr-Cys-Leu-His (SEQ ID NO:16).

An additional stable binding loop having high affinity for fibrin has the structure: Cys-Tyr-X₃-Ser-Tyr-X₆—X₇—X₈—X₉-Cys (SEQ ID NO: 17), wherein
X₃ is Asn or Asp;
X₆ is Gly or Tyr;
X₇ is His or Val;
X₈ is Pro or Trp; and
X₉ is Trp or Tyr.

Preferred polypeptides including this fibrin binding loop are polypeptides including the amino acid sequence:
X₁—X₂—X₃-Cys-Tyr-X₆-Ser-Tyr-X₉—X₁₀—X₁₁—X₁₂-Cys-X₁₄—X₁₅—X₁₆ (SEQ ID NO: 65), wherein
X₁ is Asn or Arg;
X₂ is His or Phe;
X₃ is Gly or Leu;
X₆ is Asn or Asp;
X₉ is Gly or Tyr;
X₁₀ is Val or His;
X₁₁ is Pro or Trp;
X₁₂ is Tyr or Trp;
X₁₄ is Asp or Ser;
X₁₅ is Tyr or His; and
X₁₆ is Ser or His.
   Preferred polypeptides of this formula comprise the sequence:
Asn-His-Gly-Cys-Tyr-Asn-Ser-Tyr-Gly-Val-Pro-Tyr-Cys-Asp-Tyr-Ser (SEQ ID NO: 18), or
Arg-Phe-Leu-Cys-Tyr-Asp-Ser-Tyr-Tyr-His-Trp-Trp-Cys-Ser-His-His (SEQ ID NO: 19).

A further stable binding loop having high affinity for fibrin has the structure: Cys-Pro-Tyr-Xaa-Leu-Cys (SEQ ID NO: 20), where Xaa is Asp or Gly. Preferred polypeptides including this fibrin binding loop are polypeptides including the amino acid sequence:
X₁—X₂-Cys-Pro-Tyr-X₆-Leu-Cys-X₉—X₁₀—X₁₁ (SEQ ID NO: 66), wherein
X₁ is Trp, Phe, His, or Tyr;
X₂ is His, Asp, or Glu;
X₆ is Asp, Gly, or Ala;
X₉ is His, Phe, Tyr, or Trp;
X₁₀ is Ile, Leu, or Val; and
X₁₁ is Asn, Gln, Ile, Leu, or Val.

Preferred polypeptides of this group include:
Trp-Phe-His-Cys-Pro-Tyr-Asp-Leu-Cys-His-Ile-Leu (SEQ ID NO: 21),
Gln-Trp-Glu-Cys-Pro-Tyr-Gly-Leu-Cys-Trp-Ile-Gln (SEQ ID NO: 22),
Gly-Phe-His-Cys-Pro-Tyr-Asp-Leu-Cys-His-Ile-Leu (SEQ ID NO: 23),
Phe-His-Cys-Pro-Tyr-Asp-Leu-Cys-His-Ile-Leu (SEQ ID NO: 24),
His-Cys-Pro-Tyr-Asp-Leu-Cys-His-Ile-Leu (SEQ ID NO: 25),
Phe-His-Cys-Pro-Tyr-Asp-Leu-Cys-His-Ile (SEQ ID NO: 26),
Trp-Glu-Cys-Pro-Tyr-Gly-Leu-Cys-Trp-Ile-Gln (SEQ ID NO: 27),
Glu-Cys-Pro-Tyr-Gly-Leu-Cys-Trp-Ile-Gln (SEQ ID NO: 28), and
Trp-Glu-Cys-Pro-Tyr-Gly-Leu-Cys-Trp-Ile (SEQ ID NO: 29).

Another aspect of the present invention relates to modifications of the foregoing polypeptides to provide fibrin specific imaging agents by radiolabeling, enzymatic labeling, or labeling with MR paramagnetic chelates or microparticles; incorporation into ultrasound bubbles, microparticles, microspheres, emulsions, or liposomes; or additions including optical dyes.

In another aspect of the present invention, methods for isolating fibrin binding moieties are provided. Such methods will be useful for isolating additional reagents for detection, localization, quantification, and treatment of thrombi.

In another aspect of the invention, methods of detecting fibrin-containing patho-physiologies, including thrombi, are provided, and methods for treating thrombotic diseases are provided.

In another aspect of the invention, therapeutic agents comprising a combination, conjugation or fusion of a thrombolytic agent or other therapeutic with a fibrin binding moiety according to the invention are provided. Such compositions will be useful in the treatment of thrombus associated diseases and conditions.

In another aspect of the invention, recombinant bacteriophage displaying fibrin binding polypeptides on their surfaces are also provided. Such phage are useful as screening reagents and reagents for detecting fibrin.

These and other aspects of the present invention will become apparent with reference to the following detailed description.

### DEFINITIONS

In the following sections, the term “recombinant” is used to describe non-naturally altered or manipulated nucleic acids, host cells transfected with exogenous nucleic acids, or polypeptides expressed non-naturally, through manipulation of isolated DNA and transformation of host cells. Recombinant is a term that specifically encompasses DNA molecules which have been constructed in vitro using genetic engineering techniques, and use of the term “recombinant” as an adjective to describe a molecule, construct, vector, cell, polypeptide or polynucleotide specifically excludes naturally occurring such molecules, constructs, vectors, cells, polypeptides or polynucleotides.

As used herein, the term “fibrin-derived polypeptide” refers to any subcomponent of fibrin or fragment of fibrin that is immunologically cross-reactive with fibrin, including immunologically reactive fragments of the protein.

The term “bacteriophage” is defined as a bacterial virus containing a DNA core and a protective shell built up by the aggregation of a number of different protein molecules. The terms “bacteriophage” and “phage” are used herein interchangeably.

The term “polypeptide” is used to refer to a compound of two or more amino acids joined through the main chain (as opposed to side chain) by a peptide amide bond (—C(:O)NH—)

The term “binding moiety” as used herein refers to any molecule capable of forming a binding complex with another molecule. “Fibrin binding moiety” is a binding moiety that forms a complex with a clot, soluble or insoluble fibrin, or a soluble or insoluble fragment of fibrin having a structure or characteristic exhibited by fibrin but not fibrinogen. Included among such soluble or insoluble fragments of fibrin are fragments defined as “fibrin-derived” polypeptides. Fibrin-derived polypeptides, for the purposes of this invention will be used as a collective term for the DD, DD-dimer, and DD(E) polypeptides described herein. Such fibrin-derived polypeptides are typically generated by proteolytic treatment of crosslinked fibrin but retain structural features unique to fibrin. Specific examples of fibrin binding moieties are the polypeptides described herein (including, for example, SEQ ID NOs: 1-29), hybrid and chimeric polypeptides incorporating such polypeptides, and recombinant cells or bacteriophage displaying any of such polypeptides. Also included within the definition of fibrin binding moieties are polypeptides which are modified as disclosed herein. Specific examples of modifications are C- or N-terminal amino acid substitutions or elongations, e.g., for the purpose of linking the binding moiety to a detectable imaging label or other substrate, examples of which include, e.g., addition of a polyhistidine “tail” in order to assist in purification; substitution of one up to several amino acids in order to obliterate an enzyme cleavage site; the use of N-terminal or C-terminal modifications or linkers, such as polyglycine or polylysine segments; alterations to include functional groups, notably hydrazide (—NH—NH₂) functionalities, to assist in immobilization of binding peptides according to this invention on solid supports; and the like. In addition to the detectable labels described further herein, other suitable substrates for the fibrin binding polypeptides include a thrombolytic agent or enzyme (e.g., tPA, plasmin, streptokinase, urokinase, hirudin), a liposome (e.g., loaded with thrombolytic agent, an ultrasound appropriate gas or both), or a solid support, well, plate, bead, tube, slide, filter, or dish. All such modified fibrin binding moieties are also considered fibrin binding moieties so long as they retain the ability to bind fibrin or fibrin-derived polypeptides.

The terms “DD”, “DD dimer”, and “DD(E)” refer to fibrin subcomponents typically generated by proteolytic degradation of fibrin with plasmin or trypsin. The terms “DD” and “DD dimer” both refer to the glutaminase crosslinked D domains of adjacent fibrin monomers, about 180 kDa in molecular weight. The term “DD dimer” encompasses the C-terminal portion of fibrin, including roughly α(111-197), β(134-461) and γ(88-406) in the human fibrinogen sequence. The term “DD(E)” refers to a complex of DD with the central E domain of fibrin, about 60 kDa in molecular weight, and roughly includes α(111-197), β(134-461), γ(88-406), α(17-78), β(15-122) and γ(1-62) in the human fibrinogen sequence. Since “DD” and “DD(E)” are products of proteolysis of fibrin, there may be some slight heterogeneity in their composition, depending on the mode of protease digestion and their subsequent isolation. (See, Olexa et al., Biochemistry, 20: 6139-6145 (1981); Moskowitz and Budzynski, Biochemistry, 33: 12937-12944 (1994); Spraggon et al., Nature, 389: 455-462 (1997); and references therein.)

The term “binding” refers to the determination by standard assays, including those described herein, that a binding moiety recognizes and binds reversibly to a given target. Such standard assays include equilibrium dialysis, gel filtration, and the monitoring of spectroscopic changes that result from binding.

The term “specificity” refers to a binding moiety having a higher binding affinity for one target over another. The term “fibrin specificity” refers to a fibrin binding moiety having a higher affinity for fibrin over fibrinogen. Fibrin specificity may be characterized by a dissociation constant (K_{d}) or an association constant (Kₐ) for the two tested materials.

The term “metal chelate” as used herein refers to a physiologically compatible compound consisting of one or more cyclic or acyclic multidentate organic ligands complexed to one or more paramagnetic metal ions with atomic numbers 21-29, 42, 44, or 57-83.

The term “1/T₁” as used herein refers to the longitudinal relaxation rate of water protons reversibly bound to or near in space to the paramagnetic chelate.

The term “1/T₂” as used herein refers to the transverse relaxation rate of water protons reversibly bound to or near in space to the paramagnetic chelate.

The term “R₁ or R₂” as used herein refers to relaxivity, a measure of a chelate's ability to increase the relaxation rates 1/T₁ or 1/T₂, respectively, per mM of metal ion.

The term “capable of complexing a paramagnetic metal” as used herein refers to the chemical groups on a chelator which have the ability to complex a paramagnetic metal by non-covalent forces. As used herein, the phrase “the chelator complexes the metal” means an aggregate of the chelator and metal ion held together by non-covalent forces. The term “coupled” as used herein broadly includes any attachment of the chelator to the peptide.

The term “patient” as used herein refers to any mammal, especially humans.

The term “pharmaceutically acceptable” carrier or adjuvant refers to a non-toxic carrier or adjuvant that may be administered to a patient, together with a compound of this invention, and which does not destroy the pharmacological activity thereof.

The term “ingredients” refers to any excipient or excipients, including pharmaceutical ingredients or excipients.

### DETAILED DESCRIPTION OF THE INVENTION

The present invention provides novel binding moieties for fibrin. Such binding moieties make possible the efficient detection, imaging and localization of fibrin or fibrin-derived peptides in a solution or system that contains fibrin or fibrin-derived polypeptides. In particular, the binding moieties of this invention, when appropriately labeled, are useful for detecting, imaging and localizing fibrin-containing thrombi or other fibrin specific pathophysiologies, and can thus be used to form a variety of diagnostic and therapeutic agents for diagnosing and treating thrombotic disease. The preferred binding moieties of the present invention bind fibrin and/or fibrin-derived polypeptides with high affinity, i.e., acting at low, physiologically relevant concentrations, comparable to known anti-fibrin antibodies and other fibrin-binding proteins.

Specific fibrin binding polypeptides according to the present invention were isolated initially by screening of phage display libraries, that is, populations of recombinant bacteriophage transformed to express an exogenous peptide loop on their surface. In order to isolate new polypeptide binding moieties for a particular target, such as fibrin, screening of large peptide libraries, for example using phage display techniques, is especially advantageous, in that very large numbers (e.g., 5×10⁹) of potential binders can be tested and successful binders isolated in a short period of time.

In order to prepare a phage library of potential polypeptides to screen for binding moieties such as fibrin binding peptides, a candidate binding domain is selected to serve as a structural template for the peptides to be displayed in the library. The phage library is made up of a multiplicity of analogues of the parental domain or template. The binding domain template may be a naturally occurring or synthetic protein, or a region or domain of a protein. The binding domain template may be selected based on knowledge of a known interaction between the binding domain template and fibrin, but this is not critical. In fact, it is not essential that the domain selected to act as a template for the library have any affinity for the target at all: Its purpose is to provide a structure from which a multiplicity (library) of similarly structured polypeptides (analogues) can be generated, which multiplicity of analogues will hopefully include one or more analogues that exhibit the desired binding properties (and any other properties screened for).

In selecting the parental binding domain or template on which to base the variegated amino acid sequences of the library, the most important consideration is how the variegated peptide domains will be presented to the target, i.e., in what conformation the peptide analogues will come into contact with the target. In phage display methodologies, for example, the analogues will be generated by insertion of synthetic DNA encoding the analogues into phage, resulting in display of the analogue on the surfaces of the phage. Such libraries of phage, such as M13 phage, displaying a wide variety of different polypeptides, can be prepared using techniques as described, e.g., in Kay et al., Phage Display of Peptides and Proteins: A Laboratory Manual (Academic Press, Inc., San Diego 1996) and U.S. Pat. No. 5,223,409 (Ladner et al.), incorporated herein by reference.

For formation of phage display libraries, it is preferred to use a structured polypeptide as the binding domain template, as opposed to an unstructured, linear peptide. Mutation of surface residues in a protein will usually have little effect on the overall structure or general properties (such as size, stability, and temperature of denaturation) of the protein; while at the same time mutation of surface residues may profoundly affect the binding properties of the protein. The more tightly a polypeptide segment is constrained, the less likely it is to bind to any particular target; however if the polypeptide does bind, the binding is likely to be of higher affinity and of greater specificity. Thus, it is preferred to select a parental domain and, in turn, a structure for the potential polypeptide binders, that is constrained within a framework having some degree of rigidity. In isolating the specific polypeptides according to this invention, a library designated TN7 (having 5×10⁹ amino acid sequence diversity) was used, which library was constructed for expression of diversified polypeptides on M13 phage. The TN7 library was constructed to display a single polypeptide binding loop contained in an 11-amino acid template. The TN7 library utilized a template sequence of Xaa-Xaa-Cys-Xaa-Xaa-Xaa-Xaa-Xaa-Cys-Xaa-Xaa (SEQ ID NO: 30). Additional libraries were also screened which displayed a smaller (TN6-6) and a larger (TN10-9) polypeptide binding loop.

Such small binding loop peptides offer several advantages over large proteins: First, the mass per binding site is reduced, e.g., such highly stable and low molecular weight polypeptide domains can show much higher binding per gram than do antibodies (150 kDa) or single-chain antibodies (30 kDa). Second, the possibility of non-specific binding is reduced because there is less surface available. Third, small proteins or polypeptides can be engineered to have unique tethering sites such as terminal polylysine segments in a way that is impracticable for larger proteins or antibodies. Fourth, a constrained polypeptide structure is more likely to retain its functionality when transferred with the structural domain intact from one framework to another, that is, the binding domain structure is likely to be transferable from the framework used for presentation in a library (e.g., displayed on a phage) to an isolated protein removed from the presentation framework or immobilized on a chromatographic substrate.

The TN7 library was created by making a designed series of mutations or variations within a coding sequence for the polypeptide template, each mutant sequence encoding a binding loop analogue corresponding in overall structure to the template except having one or more amino acid variations in the sequence of the template. The novel variegated (mutated) DNA provides sequence diversity, and each transformant phage displays one variant of the initial template amino acid sequence encoded by the DNA, leading to a phage population (library) displaying a vast number of different but structurally related amino acid sequences. The amino acid variations are expected to alter the binding properties of the binding loop or domain without significantly altering its structure, at least for most substitutions. It is preferred that the amino acid positions that are selected for variation (variable amino acid positions) will be surface amino acid positions, that is, positions in the amino acid sequence of the domains which, when the domain is in its most stable conformation, appear on the outer surface of the domain (i.e., the surface exposed to solution). Most preferably the amino acid positions to be varied will be adjacent or close together, so as to maximize the effect of substitutions.

As indicated previously, the techniques discussed in Kay et al., Phage Display of Peptides and Proteins: A Laboratory Manual (Academic Press, Inc., San Diego 1996) and U.S. Pat. No. 5,223,409 are particularly useful in preparing a library of potential binders corresponding to the selected parental template. The TN7 library was prepared according to such techniques, and it was screened for fibrin binding polypeptides against an immobilized fibrin target (e.g., DD(E) fibrin).

In a typical screen, a phage library is contacted with and allowed to bind the target, in this case, fibrin or a particular subcomponent, such as DD(E), presenting structures unique to the polymerized form of fibrin found in clots. To facilitate separation of binders and non-binders, it is convenient to immobilize the target on a solid support. Since fibrin is already insoluble, it is readily adaptable to phage screening. Soluble targets such as DD(E), on the other hand, must be immobilized by chemical modification. Phage bearing a target-binding moiety form a complex with the target on the solid support whereas non-binding phage remain in solution and may be washed away with excess buffer. Bound phage are then liberated from the target by changing the buffer to an extreme pH (pH 2 or pH 10), changing the ionic strength of the buffer, adding denaturants, or other known means. The recovered phage may then be amplified through infection of bacterial cells and the screening process repeated with the new pool that is now depleted in non-binders and enriched in binders. The recovery of even a few binding phage is sufficient to carry the process to completion. After a few rounds of selection, the gene sequences encoding the binding moieties derived from selected phage clones in the binding pool are determined by conventional methods, described below, revealing the peptide sequence that imparts binding affinity of the phage to the target. When the selection process works, the sequence diversity of the population falls with each round of selection until only good binders remain. The sequences converge on a small number of related binders, typically 10-50 out of the more than 10 million original candidates. An increase in the number of phage recovered at each round of selection, and of course, the recovery of closely related sequences are good indications that convergence of the library has occurred in a screen. After a set of binding polypeptides is identified, the sequence information may be used to design other secondary phage libraries, biased for members having additional desired properties.

After analysis of the sequences isolated from the library screening, a family of particular fibrin binders was defined. In addition, important consensus motifs were observed. The following sequences conforming to the TN7 template were found to bind a fibrin target:
Arg-Ser-Cys-Asn-Tyr-Tyr-Gly-Thr-Cys-Leu-His (SEQ ID NO:5);
His-Asp-Cys-Gln-Tyr-Tyr-Gly-Thr-Cys-Leu-His (SEQ ID NO:6);
Phe-Ala-Cys-His-Tyr-Tyr-Gly-Thr-Cys-Leu-His (SEQ ID NO:7);
Arg-Pro-Cys-Asp-Tyr-Tyr-Gly-Thr-Cys-Phe-Asp (SEQ ID NO:8);
Leu-Pro-Cys-Asp-Tyr-Tyr-Gly-Thr-Cys-Leu-Asp (SEQ ID NO:9);
Phe-Ser-Cys-Trp-Tyr-Ser-Leu-His-Cys-His-Arg (SEQ ID NO:10);
Asp-Pro-Cys-Ser-Tyr-Tyr-Gly-Thr-Cys-Leu-His (SEQ ID NO:11);
Leu-Pro-Cys-Ser-Tyr-Tyr-Gly-Thr-Cys-Leu-His (SEQ ID NO:12);
Leu-Ser-Cys-Asp-Tyr-Tyr-Gly-Thr-Cys-Leu-Arg (SEQ ID NO:13);
Leu-Ala-Cys-His-Tyr-Tyr-Gly-Thr-Cys-Leu-His (SEQ ID NO:14);
Asp-Gly-Cys-His-Tyr-Tyr-Gly-Thr-Cys-Leu-His (SEQ ID NO:15);
Arg-Pro-Cys-Asn-Tyr-Tyr-Gly-Thr-Cys-Leu-His (SEQ ID NO:16).

In addition, substitutions and modifications made to fibrin binding polypeptides thus identified revealed additional polypeptides of similar structure also binding to fibrin or fibrin-derived substrates (see Examples, infra.)

This series of fibrin binding polypeptides defines a family of polypeptides including the amino acid sequence:
1. X₁—X₂Cys-X₄—X₅-Tyr-X₇—X₈-Cys-X₁₀—X₁₁ (SEQ ID NO:1), wherein
   X₁ is Arg, Asp, His, Leu, or Phe;
   X₂ is Ala, Asp, Gly, Pro, or Ser;
   X₄ is Ala, Glu, Phe, Gly, Ile, Lys, Leu, Met, Arg, Thr, Val, Tyr, Asn, Asp, Gln, His, Ser, or Trp;
   X₅ is Ala, Tyr, Phe, or Ser;
   X₇ is Gly, Ala, or DAla;
X₈ is Thr, Val, or Ser;
X₁₀ is His, Leu, or Phe;
X₁₁ is Arg, Asp, or His.

The cysteine residues of the polypeptide are believed to form a disulfide bond, which causes the polypeptide to form a stable loop or cyclic structure under non-reducing conditions. Thus, the invention relates to the discovery of a fibrin binding loop comprising a polypeptide comprising the amino acid sequence: Cys-X₂—X₃-Tyr-X₅—X₆-Cys (SEQ ID NO: 2), wherein
X₂ is Ala, Glu, Phe, Gly, Ile, Lys, Leu, Met, Arg, Thr, Val, Tyr, Asn, Asp, Gln, His, Ser, or Trp;
X₃ is Ser, Phe, Ala, or Tyr;
X₅ is Gly, Ala, or DAla; and
X₆ is Thr, Val, or Ser.

A particularly strong tetramer motif was observed having the sequence Tyr-Tyr-Gly-(Thr/Ser/Val). The recurrent Tyr-Tyr-Gly-Thr motif observed in almost all of the specific isolated fibrin binding polypeptides led to selection of the particularly preferred embodiments herein: a fibrin binding loop comprising a polypeptide including the amino acid sequence: Cys-Xaa-Tyr-Tyr-Gly-Thr-Cys (SEQ ID NO:3), where Xaa is Asn, Asp, Gln, His, Ser or Trp; and a fibrin binding moiety comprising a polypeptide including the amino acid sequence Tyr-Tyr-Gly-Thr.

Screening of additional phage libraries, TN10-9 and TN6-6, led to the isolation of fibrin binding polypeptides exhibiting additional fibrin binding loop structures. The TN10-9 library led to the definition of a 10-member fibrin binding loop having the formula:
Cys-Tyr-X₃-Ser-Tyr-X₆—X₇—X₈-Cys (SEQ ID NO: 17), wherein
X₃ is Asn or Asp;
X₆ is Gly or Tyr;
X₇ is His or Val;
X₈ is Pro or Trp; and
X₉ is Trp or Tyr.

Preferred polypeptides including this fibrin binding loop are polypeptides including the amino acid sequence:
X₁—X₂—X₃-Cys-Tyr-X₆-Ser-Tyr-X₉—X₁₀—X₁₁—X₁₂-Cys-X₁₄—X₁₅—X₁₆ (SEQ ID NO: 65), wherein
X₁ is Asn or Arg;
X₂ is His or Phe;
X₃ is Gly or Leu;
X₆ is Asn or Asp;
X₉ is Gly or Tyr;
X₁₀ is Val or His;
X₁₁ is Pro or Trp;
X₁₂ is Tyr or Trp;
X₁₄ is Asp or Ser;
X₁₅ is Tyr or His; and
X₁₆ is Ser or His.

Preferred examples of such polypeptides are: Asn-His-Gly-Cys-Tyr-Asn-Ser-Tyr-Gly-Val-Pro-Tyr-Cys-Asp-Tyr-Ser (SEQ ID NO: 18), and Arg-Phe-Leu-Cys-Tyr-Asp-Ser-Tyr-Tyr-His-Trp-Trp-Cys-Ser-His-His (SEQ ID NO: 19).

The TN6-6 library led to the definition of a 6-member fibrin binding loop having the formula: Cys-Pro-Tyr-Xaa-Leu-Cys (SEQ ID NO: 20), where Xaa is Asp or Gly. Preferred polypeptides including this fibrin binding loop are polypeptides including the amino acid sequence:
hd 1—X₂-Cys-Pro-Tyr-X₆-Leu-Cys-X₉—X₁₀—X₁₁ (SEQ ID NO: 66), wherein
X₁ is Trp, Phe, His, or Tyr;
X₂ is His, Asp, or Glu;
X₆ is Asp, Gly, or Ala;
X₉ is His, Phe, Tyr, or Trp;
X₁₀ is Ile, Leu, or Val; and
X₁₁ is Asn, Gln, Ile, Leu, or Val.

Particularly preferred examples of such polypeptides include:
Trp-Phe-His-Cys-Pro-Tyr-Asp-Leu-Cys-His-Ile-Leu (SEQ ID NO: 21),
Gln-Trp-Glu-Cys-Pro-Tyr-Gly-Leu-Cys-Trp-Ile-Gln (SEQ ID NO: 22),
Gly-Phe-His-Cys-Pro-Tyr-Asp-Leu-Cys-His-Ile-Leu (SEQ ID NO: 23),
Phe-His-Cys-Pro-Tyr-Asp-Leu-Cys-His-Ile-Leu (SEQ ID NO: 24),
His-Cys-Pro-Tyr-Asp-Leu-Cys-His-Ile-Leu (SEQ ID NO: 25),
Phe-His-Cys-Pro-Tyr-Asp-Leu-Cys-His-Ile (SEQ ID NO: 26),
Trp-Glu-Cys-Pro-Tyr-Gly-Leu-Cys-Trp-Ile-Gln (SEQ ID NO: 27),
Glu-Cys-Pro-Tyr-Gly-Leu-Cys-Trp-Ile-Gln (SEQ ID NO: 28), and
Trp-Glu-Cys-Pro-Tyr-Gly-Leu-Cys-Trp-Ile (SEQ ID NO: 29).

Direct synthesis of the peptides of the invention may be accomplished using conventional techniques, including solid-phase peptide synthesis, solution-phase synthesis, etc. Solid-phase synthesis is preferred. In solid-phase synthesis, for example, a suitably protected amino acid residue is attached through its carboxyl group to a derivatized, insoluble polymeric support, such as cross-linked polystyrene or polyamide resin. “Suitably protected” refers to the presence of protecting groups on both the cc-amino group of the amino acid, and on any side chain functional groups. Side chain protecting groups are generally stable to the solvents, reagents and reaction conditions used throughout the synthesis and are removable under conditions which do not affect the final peptide product. Stepwise synthesis of the polypeptide is carried out by the removal of the N-protecting group from the initial amino acid, and coupling thereto of the carboxyl end of the next amino acid in the sequence of the polypeptide. This amino acid is also suitably protected. The carboxyl of the incoming amino acid can be activated to react with the N-terminus of the bound amino acid by formation into a reactive group such as formation into a carbodiimide, a symmetric acid anhydride or an “active ester” group such as hydroxybenzotriazole or pentafluorophenyl esters. The preferred solid-phase peptide synthesis methods include the BOC method which utilizes tert-butyloxycarbonyl as the α-amino protecting group, and the FMOC method which utilizes 9-fluorenylmethloxycarbonyl to protect the α-amino of the amino acid residues, both methods of which are well-known by those of skill in the art. See, Stewart et al., Solid-Phase Peptide Synthesis (1989), W. H. Freeman Co., San Francisco; Merrifield, J. Am. Chem. Soc., 85:2149-2154 (1963); Bodanszky and Bodanszky, The Practice of Peptide Synthesis (Springer-Verlag, New York 1984), incorporated herein by reference.

Polypeptides according to the invention may also be prepared commercially by companies providing peptide synthesis as a service (e.g., BACHEM Bioscience, Inc., King of Prussia, Pa.; Quality Controlled Biochemicals, Inc., Hopkinton, Mass.).

Automated peptide synthesis machines, such as manufactured by Perkin-Elmer Applied Biosystems, also are available.

The polypeptide compound is preferably purified once it has been isolated or synthesized by either chemical or recombinant techniques. For purification purposes, there are many standard methods that may be employed including reversed-phase high-pressure liquid chromatography (HPLC) using an alkylated silica column such as C₄-, C₈- or C₁₈-silica. A gradient mobile phase of increasing organic content is generally used to achieve purification, for example, acetonitrile in an aqueous buffer, usually containing a small amount of trifluoroacetic acid. Ion-exchange chromatography can also be used to separate peptides based on their charge. The degree of purity of the polypeptide may be determined by various methods, including identification of a major large peak on HPLC. A polypeptide that produces a single peak that is at least 95% of the input material on an HPLC column is preferred. Even more preferable is a polypeptide that produces a single peak that is at least 97%, at least 98%, at least 99% or even 99.5% of the input material on an HPLC column.

In order to ensure that the peptide obtained using any of the techniques described above is the desired peptide for use in compositions of the present invention, analysis of the peptide composition may be carried out. Such composition analysis may be conducted using high resolution mass spectrometry to determine the molecular weight of the peptide. Alternatively, the amino acid content of the peptide can be confirmed by hydrolyzing the peptide in aqueous acid, and separating, identifying and quantifying the components of the mixture using HPLC, or an amino acid analyzer. Protein sequenators, which sequentially degrade the peptide and identify the amino acids in order, may also be used to determine definitely the sequence of the peptide.

The fibrin binding polypeptides of the invention may be conformationally restrained by disulfide linkages between the two cysteine residues in their sequence. This conformational restraint ensures that the peptides have a binding structure that contributes to the peptides' affinity for fibrin and their specificity for fibrin over fibrinogen. Other methods for constraining peptides which would retain a similar conformation and fibrin specificity for the peptide have been described in the art and are contemplated herein, including the substitution of one or more of the cysteine residues with non-naturally occurring amino acids or peptidomimetics for the purpose of forming a more stable or conformationally preferred linkage between the two positions on the peptide. All such modified fibrin binding moieties are also considered fibrin binding moieties so long as they retain the ability to bind fibrin or fibrin-derived polypeptides. Non-cyclized, or linear, versions of the peptides may also retain moderate binding ability and specificity for fibrin and could also be employed in the present invention.

Homologues of the fibrin binding polypeptides described herein may be formed by substitution, addition or deletion of one or more amino acids employing methods well known in the art and for particular purposes known in the art. Such homologous polypeptides will be understood to fall within the scope of the present invention so long as the substitution, addition or deletion of amino acids does not eliminate its ability to bind fibrin. The term “homologous”, as used herein, refers to the degree of sequence similarity between two polymers (i.e., polypeptide molecules or nucleic acid molecules). When the same nucleotide or amino acid residue occupies a sequence position in the two polymers under comparison, then the polymers are homologous at that position. For example, if the amino acid residues at 60 of 100 amino acid positions in two polypeptide sequences match or are homologous then the two sequences are 60% homologous. The homology percentage figures referred to herein reflect the maximal homology possible between the two polymers, i.e., the percent homology when the two polymers are so aligned as to have the greatest number of matched (homologous) positions. Polypeptide homologues within the scope of the present invention will be at least 80% and preferably greater than 90% homologous to at least one of the fibrin binding sequences disclosed herein.

Fibrin binding polypeptides according to the present invention also may be produced using recombinant DNA techniques, utilizing nucleic acids (polynucleotides) encoding the polypeptides according to this invention and then expressing them recombinantly, i.e., by manipulating host cells by introduction of exogenous nucleic acid molecules in known ways to cause such host cells to produce the desired fibrin binding polypeptides. Recombinant production of short peptides such as those described herein may not be practical in comparison to direct synthesis, however recombinant means of production may be very advantageous where a fibrin binding motif of this invention are desired to be incorporated in a hybrid polypeptide or fusion protein.

The polynucleotides of the present invention may be in the form of RNA or in the form of DNA, which DNA includes cDNA and synthetic DNA. The DNA may be double-stranded or single-stranded, and if single stranded may be the coding strand or non-coding (anti-sense) strand. The coding sequences for fibrin binding polypeptides according to the present invention may be manipulated or varied in known ways to yield alternative coding sequences that, as a result of the redundancy or degeneracy of the genetic code, encode the same polypeptide.

The polypeptides and polynucleotides of the present invention are preferably provided in an isolated form, and preferably are purified to homogeneity. For the purposes described herein, homogeneity is considered to be a preparation which results in 99.5% of the input material obtained in a single peak on an HPLC column.

Where recombinant production of fibrin binding polypeptides is desired, the present invention also contemplates vectors that include polynucleotides of the present invention, host cells that are genetically engineered with vectors of the invention, and recombinant polypeptides produced by culturing such genetically engineered host cells. Host cells are genetically engineered (transduced or transformed or transfected) with the vectors of this invention which may be, for example, a cloning vector or an expression vector. The vector may be, for example, in the form of a plasmid, a viral particle, a phage, etc. The engineered host cells can be cultured in conventional nutrient media modified as appropriate for activating promoters, selecting transformants or amplifying the fibrin binder-encoding polynucleotides. The culture conditions, such as temperature, pH and the like, are those suitable for use with the host cell selected for expression and will be apparent to the skilled practitioner in this field. The polynucleotide may be included in any one of a variety of expression vectors for expressing a polypeptide. Such vectors include chromosomal, nonchromosomal and synthetic DNA sequences, e.g., derivatives of SV40; bacterial plasmids; phage DNA; baculovirus; yeast plasmids; vectors derived from combinations of plasmids and phage DNA, viral DNA such as vaccinia, adenovirus, fowl pox virus, and pseudorabies. However, any other vector may be used as long as it is replicable and viable in the host. The appropriate DNA sequence may be inserted into the vector by a variety of procedures. In general, the DNA sequence is inserted into an appropriate restriction endonuclease site(s) by procedures known in the art. Such procedures and others are within the capability of those skilled in the art.

The DNA sequence in the expression vector is operatively linked to an appropriate expression control sequence(s) (promoter) to direct mRNA synthesis. As representative examples of such promoters, there may be mentioned LTR or SV40 promoter, the E. coli. lac or trp, the phage lambda P_{L} promoter and other promoters known to control expression of genes in prokaryotic or eukaryotic cells or their viruses. The expression vector also contains a ribosome binding site for translation initiation and a transcription terminator. The vector may also include appropriate sequences for amplifying expression. In addition, expression vectors preferably will contain one or more selectable marker genes to provide a phenotypic trait for selection of transformed host cells, such as dihydrofolate reductase or neomycin resistance for eukaryotic cell culture, or such as tetracycline or ampicillin resistance for bacterial cell cultures such as E. coli.

The vector containing the appropriate DNA sequence as hereinabove described, as well as an appropriate promoter or control sequence, may be employed to transform an appropriate host to permit the host to express the protein. As representative examples of appropriate host cells, there may be mentioned bacterial cells, such as E. coli, Streptomyces, Salmonella typhimurium; flingal cells, such as yeast; insect cells such as Drosophila and Sf9; animal cells such as CHO, COS or Bowes melanoma; plant cells, etc. The selection of an appropriate host for this type of fibrin binder production is also within the capability of those skilled in the art from the teachings herein. Many suitable vectors and promoters useful in expression of proteins according to this invention are known to those of skill in the art, and many are commercially available. The following vectors are provided by way of example. Bacterial: pQE70, pQE60, pQE-9 (Qiagen), pbs, pD10, phagescript, psiX174, pbluescript SK, pbsks, pNH8A, pNH16a, pNH18A, pNH46A (Stratagene); ptrc99a, pKK223-3, pKK233-3, pDR540, pRIT5 (Pharmacia). Eukaryotic: pWLNEO, pSV2CAT, pOG44, pXT1, pSG (Stratagene) pSVK3, pBPV, pMSG, pSVL (Pharmacia). Any other plasmid or vector may be used as long as it is replicable and viable in the selected host cell.

Introduction of the vectors into the host cell can be effected by any known method, including calcium phosphate transfection, DEAE-Dextran mediated transfection, or electroporation (see Davis et al., Basic Methods in Molecular Biology, (1986)).

In the practice of the present invention, a determination of the affinity of the fibrin binding moiety for fibrin relative to fibrinogen is a useful measure, and is referred to as specificity for fibrin. Standard assays for quantitating binding and determining affinity include equilibrium dialysis, equilibrium binding, gel filtration, or the monitoring of numerous spectroscopic changes (such as fluorescence) that may result from the interaction of the binding moiety and its target. These techniques measure the concentration of bound and free ligand as a function of ligand (or protein) concentration. The concentration of bound polypeptide ([Bound]) is related to the concentration of free polypeptide ([Free]) and the concentration of binding sites for the polypeptide, i.e., on fibrin, (N), as described in the following equation:
[Bound]=N×[Free]/((1/Kₐ)+[Free]).
A solution of the data to this equation yields the association constant, Kₐ, a quantitative measure of the binding affinity. The association constant, Kₐ is the reciprocal of the dissociation constant, K_{d}. The K_{d} is more frequently reported in measurements of affinity. A peptide having a K_{d} 1.5 times higher for fibrinogen than for fibrin would be considered as a weak fibrin binder. A peptide having a K_{d} 10 times greater for fibrinogen than fibrin would be a moderate fibrin binder, and a peptide having a K_{d} 100 times or more greater for fibrinogen than for fibrin would be termed highly specific for fibrin. Preferably the peptides and agents of the present invention have a K_{d} at least 1.5 times higher for fibrinogen than for fibrin, more preferably at least 10 times higher, even more preferably at least 100 times, and most preferably at least 1000 times higher. Preferred fibrin binding polypeptides have a K_{d} for fibrin in the range of 1 nanomolar (nM) to 100 micromolar (μM) and includes K_{d} values of at least 10 nM, at least 20 nM, at least 40 nM, at least 60 nM, at least 80 nM, at least 1 μM, at least 5 μM, at least 10 μM, at least 20 μM, at least 40 μM, at least 60 μM, and at least 80 μM.

The foregoing assay of fibrin affinity can be adapted to a microtiter plate format for evaluating large numbers of polypeptides. Single point concentrations can be used to quickly differentiate molecules of high fibrin specificity or binding affinity from those with low fibrin specificity or binding affinity.

An alternative, empirical approach for measuring fibrin affinity is to form a clot in tubing of a small diameter (e.g., 3 mm), then perfuse the clot with buffer or plasma containing the test polypeptide(s). The concentration of the peptide in the solution as it elutes from the clot is monitored by standard methods (e.g., HPLC separation followed by mass spectrometric detection or fluorescence labeling). Polypeptides that are well retained in the clot are identified as good binders to fibrin clots and as having a useful degree of specificity for fibrin over fibrinogen.

Uses for Fibrin Binding Polypeptides

The fibrin binding moieties according to this invention will be extremely useful for detection and/or imaging of fibrin in vitro or in vivo, and particularly for detection and/or imaging of fibrin clots. Any suitable method of assaying or imaging fibrin may be employed.

For detection of fibrin or fibrin-derived polypeptides in solution, a binding moiety according to the invention can be detectably labeled, e.g., radiolabeled or enzymatically labeled, then contacted with the solution, and thereafter formation of a complex between the binding moiety and the fibrin target can be detected. As an example, bacteriophage expressing fibrin binding polypeptides on their surface may be used for in vitro fibrin detection assays, wherein the bacteriophage are added to a solution to be tested for fibrin under conditions allowing binding to occur. A detectable phage-binding moiety, such as an anti-phage antibody that recognizes a portion of the phage coat (that is not directly involved in binding), may then be incubated with the solution containing any fibrin/phage complexes. Such detection antibodies may be labeled in known ways, e.g., with horseradish peroxidase, for visualization according to known techniques. The ternary complex between fibrin-binding phage, fibrin, and the antibody can then be detected and quantitated.

Alternatively, a sandwich-type assay may be used, wherein a fibrin binding moiety is immobilized on a solid support such as a plastic tube or well, then the solution suspected of containing fibrin or a fibrin-derived polypeptide is contacted with the immobilized binding moiety, non-binding materials are washed away, and complexed polypeptide is detected using a suitable detection reagent, such as a monoclonal antibody recognizing fibrin. The monoclonal antibody is detectable by conventional means known in the art, including being detectably labeled, e.g., radiolabeled, conjugated with an enzyme such as horseradish peroxidase and the like, or fluorescently labeled.

For detection or purification of soluble fibrin or fibrin-derived polypeptides in or from a solution, a binding moiety of the invention can be immobilized on a solid substrate such as a chromatographic support or other porous material, then the immobilized binder can be loaded or contacted with the solution under conditions suitable for formation of a binding moiety/fibrin complex. The non-binding portion of the solution can be removed and the complex may be detected, e.g., using an anti-fibrin or anti-binding moiety antibody, or the fibrin target may be released from the binding moiety at appropriate elution conditions.

The biology of fibrin and clot formation has been investigated by many researchers and continues to be an active field for research and development. Pure fibrin also may have utility as a therapeutically useful clotting agent. In furtherance of such research and development, a method of purifying bulk amounts of fibrin in pure form is desirable, and the binding moieties according to this invention are especially useful for that purpose, using the general purification methodology described above.

Thrombus Imaging

A particularly preferred use for the polypeptides according to the present invention is for creating visually readable images of thrombi, to aid in the diagnosis, monitoring and treatment of thrombus associated disorders. The fibrin binding polypeptides disclosed herein may be converted to imaging reagents for detecting thrombi by conjugating the polypeptides with a label appropriate for diagnostic detection. Preferably, a peptide exhibiting much greater specificity for fibrin than for fibrinogen is conjugated or linked to a label appropriate for the detection methodology to be employed. For example, the fibrin binder may be conjugated with a paramagnetic chelate suitable for magnetic resonance imaging (MRI), with a radiolabel suitable for x-ray imaging, with an ultrasound microsphere or liposome suitable for ultrasound detection, or with an optical imaging dye.

Suitable linkers can be substituted or unsubstituted alkyl chains, amino acid chains (e.g., polyglycine), polyethylene glycols, polyamides, and other simple polymeric linkers known in the art.

In general, the technique of using a detectably labeled fibrin binding moiety is based on the premise that the label generates a signal that is detectable outside the patient's body. When the detectably labeled fibrin binding moiety is administered to the patient suspected of having a thrombus, the high affinity of the fibrin binding moiety for fibrin in a thrombus causes the fibrin binding moiety to bind to the thrombus and accumulate label at the site of the thrombus. Sufficient time is allowed for the labeled peptide to localize at the site of the thrombus. The signal generated by the labeled peptide is detected by a scanning device which will vary according to the type of label used, and the signal is then converted to an image of the thrombus.

A. Magnetic Resonance Imaging

The fibrin binding moieties of the present invention may advantageously be conjugated with a paramagnetic metal chelate in order to form a contrast agent for use in MRI. Preferred paramagnetic metal ions have atomic numbers 21-29, 42, 44, or 57-83. This includes ions of the transition metal or lanthanide series which have one, and more preferably five or more, unpaired electrons and a magnetic moment of at least 1.7 Bohr magneton. The preferred paramagnetic metal is selected from the group consisting of Gd(III), Fe(III), Mn(II and III), Cr(III), Cu(II), Dy(III), Tb(III), Ho(III), Er(III), and Eu(III). Gd(III) is particularly preferred for MRI due to its high relaxivity and low toxicity, and the availability of only one biologically accessible oxidation state. Gd(III) chelates have been used for clinical and radiologic MR applications since 1988, and approximately 30% of MR exams currently employ a gadolinium-based contrast agent.

In order to effectively enhance NMR imaging, the complex must be capable of enhancing the relaxation rates 1/T₁ (longitudinal, or spin-lattice) and/or 1/T₂ (transverse or spin-spin) of water protons or other imaging or spectroscopic nuclei, including protons, P-31, C13, Na-23, or F-19 on other biomolecules or injected biomarkers. Relaxivities R₁ and R₂ are defined as the ability to increase 1/T₁ or 1/T₂, respectively, per mM of metal ion; units are mM⁻¹s⁻¹. For the most common form of clinical MRI, water proton MRI, relaxivity is optimal where the paramagnetic ion bound to the chelating ligand still has one or more open coordination sites for water exchange (Lauffer, Chemical Review, 87: 901-927 (1987)). However, this must be balanced with the stability of the metal chelate (vide infra) which generally decreases with increasing numbers of open coordination sites. More preferably, therefore, the complex contains only one or two open coordination sites.

Although the paramagnetic metal is used in a complexed from, toxic effects may still arise due to the dissociation of the metal ion from the complex. The organic chelating ligand should be physiologically compatible. The molecular size of the chelating ligand should be compatible with the size of the paramagnetic metal. Thus, gadolinium (III), which has a crystal ionic radius of 0.938 Å, requires a larger chelating ligand than iron (III), which has a crystal ionic radius of 0.64 Å.

In general, the degree of toxicity of a metal chelate is related to its degree of dissociation in vivo before excretion. Toxicity generally increases with the amount of free metal ion. For complexes in which kinetic stability is low, a high thermodynamic stability (a formation constant of at least 10¹⁵ M⁻¹ and more preferably at least 10²⁰ M⁻¹) is desirable to minimize disassociation and its attendant toxicity. For complexes in which kinetic stability is comparatively higher, dissociation can be minimized with a lower formation constant, i.e., 10¹⁰ M⁻¹ or higher. Toxicity is also a function of the number of open coordination sites in the complex. The fewer coordination sites, the less tendency there is, generally, for the chelating agent to release the paramagnetic substance. Preferably, therefore, the complex contains two, one or zero open coordination sites. The presence of more than two open sites in general will unacceptably increase toxicity by excessive release of the metal ion in vivo.

The practitioner will select a metal according to dose required to detect a thrombus and considering other factors such as toxicity of the metal to the subject. See, Tweedle et al., Magnetic Resonance Imaging (2nd ed.), vol. 1, Partain et al., eds. (W.B. Saunders Co. 1988), pp. 796-7. Generally, the desired dose for an individual metal will be proportional to its relaxivity, modified by the biodistribution, pharmacokinetics and metabolism of the metal. As mentioned previously, the trivalent cation, Gd³⁺ is particularly preferred for MRI contrast agents, due to its high relaxivity and low toxicity, with the further advantage that it exists in only one biologically accessible oxidation state, which minimizes undesired metabolization of the metal by a patient. Another useful metal is Cr³⁺, which is relatively inexpensive.

The organic chelator is a molecule having one or more polar groups that act as a ligand for, and complex with, a paramagnetic metal. Suitable chelators are known in the art and include acids with methylene phosphonic acid groups, methylene carbohydroxamine acid groups, carboxyethylidene groups, or carboxymethylene groups. Examples of chelators include, but are not limited to, diethylenetriaminepentaacetic acid (DTPA), 1,4,7,10-tetraazacyclotetradecane-1,4,7,10-tetraacetic acid (DOTA), ethylenediaminetetraacetic acid (EDTA), and 1,4,8,11-tetraazacyclotetradecane-1,4,8,11-tetraacetic acid (TETA). Additional chelating ligands are ethylenebis-(2-hydroxy-phenylglycine) (EHPG), and derivatives thereof, including 5-Cl-EHPG, 5Br-EHPG, 5-Me-EHPG, 5t-Bu-EHPG, and 5sec-Bu-EHPG; benzodiethylenetriamine pentaacetic acid (benzo-DTPA) and derivatives thereof, including dibenzo-DTPA, phenyl-DTPA, diphenyl-DTPA, benzyl-DTPA, and dibenzyl DTPA; bis-2 (hydroxybenzyl)-ethylene-diaminediacetic acid (HBED) and derivatives thereof; the class of macrocyclic compounds which contain at least 3 carbon atoms, more preferably at least 6, and at least two heteroatoms (O and/or N), which macrocyclic compounds can consist of one ring, or two or three rings joined together at the hetero ring elements, e.g., benzo-DOTA, dibenzo-DOTA, and benzo-NOTA, where NOTA is 1,4,7-triazacyclononane N,N′,N″-triacetic acid, benzo-TETA, benzo-DOTMA, where DOTMA is 1,4,7,10-tetraazacyclotetradecane-1,4,7, 10-tetra(methyl tetraacetic acid), and benzo-TETMA, where TETMA is 1,4,8,11-tetraazacyclotetradecane-1,4,8,11-(methyl tetraacetic acid); derivatives of 1,3-propylenediaminetetraacetic acid (PDTA) and triethylenetetraaminehexaacetic acid (TTHA); derivatives of 1,5,10-N,N′,N″-tris(2,3-dihydroxybenzoyl)-tricatecholate (LICAM) and 1,3,5-N,N′,N″-tris(2,3-dihydroxybenzoyl) aminomethylbenzene (MECAM). A preferred chelator for use in the present invention is DTPA. Examples of representative chelators and chelating groups contemplated by the present invention are described in WO 98/18496, WO 86/06605, WO 91/03200, WO 95/28179, WO 96/23526, WO 97/36619, PCT/US98/01473, PCT/US98/20182, and U.S. Pat. No. 4,899,755, all of which are hereby incorporated by reference.

In accordance with the present invention, the chelator of the MRI contrast agent is coupled to the fibrin binding moiety. The positioning of the chelate should be selected so as not to interfere with the binding affinity or specificity of the fibrin binding moiety. Preferably, the chelate will be appended either to the N terminus or the C terminus, however the chelate may also be attached anywhere within the sequence. In preferred embodiments, a chelator having a free central carboxylic acid group (e.g., DTPA-Asp(β-COOH)-OtBu) makes it easy to attach at the N-terminus of the peptide by formation of an amide bond. The chelate could also be attached at the C-terminus with the aid of a linker. Alternatively, isothiocyanate conjugation chemistry could be employed as a way of linking the appropriate isothiocyanto group bearing DTPA to a free amino group anywhere within the peptide sequence.

In general, the fibrin binding moiety can be bound directly or covalently to the metal chelator (or other detectable label), or it may be coupled or conjugated to the metal chelator using a linker, which may be, without limitation, amide, urea, acetal, ketal, double ester, carbonyl, carbamate, thiourea, sulfone, thioester, ester, ether, disulfide, lactone, imine, phosphoryl, or phosphodiester linkages; substituted or unsubstituted saturated or unsaturated alkyl chains; linear, branched, or cyclic amino acid chains of a single amino acid or different amino acids (e.g., extensions of the N- or C-terminus of the fibrin binding moiety); derivatized or underivatized polyethylene glycol, polyoxyethylene, or polyvinylpyridine chains; substituted or unsubstituted polyamide chains; derivatized or underivatized polyamine, polyester, polyethylenimine, polyacrylate, poly(vinyl alcohol), polyglycerol, or oligosaccharide (e.g., dextran) chains; alternating block copolymers; malonic, succinic, glutaric, adipic and pimelic acids; caproic acid; simple diamines and dialcohols; and other simple polymeric linkers known in the art (see, e.g., WO 98/18497, WO 98/18496). Preferably the molecular weight of the linker can be tightly controlled. The molecular weights can range in size from less than 100 to greater than 1000. Preferably the molecular weight of the linker is less than 100. In addition, it may be desirable to utilize a linker that is biodegradable in vivo to provide efficient routes of excretion for the imaging reagents of the present invention. Depending on their location within the linker, such biodegradable functionalities can include ester, double ester, amide, phosphoester, ether, acetal, and ketal functionalities.

In general, known methods can be used to couple the metal chelate and the fibrin binding moiety using such linkers. See, e.g., WO 95/28967, WO 98/18496, WO 98/18497 and discussion therein. The fibrin binding moiety can be linked through its N- or C-terminus via an amide bond, for example, to a metal coordinating backbone nitrogen of a metal chelate or to an acetate arm of the metal chelate itself. The present invention contemplates linking of the chelate on any position, provided the metal chelate retains the ability to bind the metal tightly in order to minimize toxicity. Similarly, the fibrin binding moiety may be modified or elongated in order to generate a locus for attachment to a metal chelate, provided such modification or elongation does not eliminate its ability to bind fibrin.

MRI contrast reagents prepared according to the disclosures herein may be used in the same manner as conventional MRI contrast reagents. When imaging a thrombus, certain MR techniques and pulse sequences may be preferred to enhance the contrast of the thrombus to the background blood and tissues. These techniques include (but are not limited to), for example, black blood angiography sequences that seek to make blood dark, such as fast spin echo sequences (see, e.g., Alexander et al., Magnetic Resonance in Medicine, 40(2): 298-310 (1998)) and flow-spoiled gradient echo sequences (see,e.g., Edelman et al., Radiology, 177(1): 45-50 (1990)). These methods also include flow independent techniques that enhance the difference in contrast due to the T₁ difference of contrast-enhanced thrombus and blood and tissue, such as inversion-recovery prepared or saturation-recovery prepared sequences that will increase the contrast between thrombus and background tissues. In addition, since the present invention does not significantly alter T₂, methods of T₂ preparation may also prove useful (see, e.g., Gronas et al., Journal of Magnetic Resonance Imaging, 7(4): 637-643 (1997)). Finally, magnetization transfer preparations may also improve contrast with these agents (see, e.g., Goodrich et al., Investigative Radiology, 31(6): 323-32 (1996)).

The labeled reagent is administered to the patient in the form of an injectable composition. The method of administering the MRI contrast agent is preferably parenterally, meaning intravenously, intraarterially, intrathecally, interstitially, or intracavitarilly. For imaging thrombi, intravenous or intraarterial administration is preferred. For MRI, it is contemplated that the subject will receive a dosage of contrast agent sufficient to enhance the MR signal at the site of a thrombus at least 10%. After injection with the fibrin binding moiety-containing MRI reagent, the patient is scanned in the MRI machine to determine the location of any thrombi. In therapeutic settings, upon thrombus localization, a thrombolytic can be immediately administered, if necessary, and the patient can be subsequently scanned to visualize thrombus degradation.

B. Ultrasound Imaging

When ultrasound is transmitted through a substance, the acoustic properties of the substance will depend upon the velocity of the transmissions and the density of the substance. Changes in the acoustic properties will be most prominent at the interface of different substances (solids, liquids, gases). Ultrasound contrast agents are intense sound wave reflectors because of the acoustic differences between liquid (e.g., blood) and gas-containing microbubbles, liposomes, or microspheres dissolved therein. Because of their size, ultrasound microbubbles, liposomes, microspheres, and the like may remain for a longer time in the blood stream after injection than other detectable moieties; a targeted fibrin-specific ultrasound agent therefore may demonstrate superior imaging of thrombi.

In this aspect of the invention, the fibrin binding moiety may be linked to a material which is useful for ultrasound imaging. The materials are employed to form vesicles (e.g., liposomes, microbubbles, microspheres, or emulsions) containing a liquid or gas which functions as the detectable label (e.g., an echogenic gas or material capable of generating an echogenic gas). Materials for the preparation of such vesicles include surfactants, lipids, sphingolipids, oligolipids, phospholipids, proteins, polypeptides, carbohydrates, and synthetic or natural polymeric materials. See, for further description of suitable materials and methods, WO 98/53857, WO 98/18498, WO 98/18495, WO 98/18497, WO 98/18496, and WO 98/18501.

Suitable gases include, but are not limited to, C₁₋₆ perfluorcarbon gases, SF₆, low molecular weight C₁₋₆ fluorinated or halogenated alkenes, alkynes, or cyclized versions of the same, or other suitable gases or mixtures thereof, as described in WO 97/29783, WO 98/53857, WO 98/18498, WO 98/18495, WO 98/18496, WO 98/18497, WO 98/18501, WO 98/05364, WO 98/17324. The term “gas” as used herein refers to materials that are in the gaseous state at the normal human body temperature of 37° C. The ultrasound vesicles may be used as is or stabilized with surfactants or some other stabilizing material such as emulsifying agents and/or viscosity enhancers, cryoprotectants, lyoprotectants, or bulking agents.

Since ultrasound vesicles may be larger than the other detectable labels described herein, they may be linked or conjugated with a plurality of fibrin binding moieties on their surfaces in order to increase the targeting efficiency of the agent. Attachment may be via direct covalent bond between the fibrin binding moiety and the material used to make the vesicle or via a linker, as described previously. For example, see WO 98/53857 generally for a description of the attachment of a peptide to a bifunctional PEG linker, which is then reacted with a liposome composition. See also, Lanza et al., Ultrasound in Med. & Bio., 23(6): 863-870 (1997). The targeted ultrasound vesicles may be prepared using conventional methods known in the art. Known methods include gentle shaking, rotor mixing, sonication, high pressure homogenization, high speed stirring, high shear mixing, emulsification, and colloidal mill procedures, in the presence or absence of the desired echogenic gas or gas mixture, to generate the vesicles. The desired echogenic gas may alternatively be incorporated into the vesicles by applying an atmosphere or overpressure of said gas to the vesicles (see U.S. Pat. No. 5,674,469).

Ultrasound imaging techniques which may be used in accordance with the present invention include known techniques, such as color Doppler, power Doppler, Doppler amplitude, stimulated acoustic imaging, and two- or three-dimensional imaging techniques. Imaging may be done in harmonic (resonant frequency) or fundamental modes, with the second harmonic preferred.

C. Optical Imaging, Sonoluminescence or Photoacoustic Imaging

In accordance with the present invention, a number of optical parameters may be employed to determine the location of fibrin with in vivo light imaging after injection of the subject with an optically-labeled fibrin binding moiety. Optical parameters to be detected in the preparation of an image may include transmitted radiation, absorption, fluorescent or phosphorescent emission, light reflection, changes in absorbance amplitude or maxima, and elastically scattered radiation. For example, biological tissue is relatively translucent to light in the near infrared (NIR) wavelength range of 650-1000 nm. NIR radiation can penetrate tissue up to several centimeters, permitting the use of the fibrin binding moieties of the present invention for optical imaging of fibrin in vivo.

The fibrin binding moieties may be conjugated with photolabels, such as optical dyes, including organic chromophores or fluorophores, having extensive delocalized ring systems and having absorption or emission maxima in the range of 400-1500 nm. The fibrin binding moiety may alternatively be derivatized with a bioluminescent molecule. The preferred range of absorption maxima for photolabels is between 600 and 1000 nm to minimize interference with the signal from hemoglobin. Preferably, photoabsorption labels have large molar absorptivities, e.g. >10⁵ cm⁻¹M⁻¹, while fluorescent optical dyes will have high quantum yields. Examples of optical dyes include, but are not limited to those described in WO 98/18497, WO 98/18496, WO 98/18495, WO 98/18498, WO 98/53857, WO 96/17628, WO 97/18841, WO 96/23524, WO 98/47538, and references cited therein. The photolabels may be covalently linked directly to the fibrin binding moiety or linked to the fibrin binding moiety via a linker, as described previously.

After injection of the optically-labeled fibrin binding moiety, the patient is scanned with one or more light sources (e.g., a laser) in the wavelength range appropriate for the photolabel employed in the agent. The light used may be monochromatic or polychromatic and continuous or pulsed. Transmitted, scattered, or reflected light is detected via a photodetector tuned to one or multiple wavelengths to determine the location of fibrin in the subject. Changes in the optical parameter may be monitored over time to detect accumulation of the optically-labeled reagent at the site of the thrombus. Standard image processing and detecting devices may be used in conjunction with the optical imaging reagents of the present invention.

The optical imaging reagents described above may also be used for acousto-optical or sonoluminescent imaging performed with optically-labeled imaging agents (see, U.S. Pat. No. 5,171,298, WO 98/57666, and references therein). In acousto-optical imaging, ultrasound radiation is applied to the subject and affects the optical parameters of the transmitted, emitted, or reflected light. In sonoluminescent imaging, the applied ultrasound actually generates the light detected. Suitable imaging methods using such techniques are described in WO 98/57666.

D. Nuclear Imaging (Radionuclide Imaging).

The fibrin binding moieties may be conjugated with a radionuclide reporter appropriate for scintigraphy, SPECT, or PET imaging. For use as a PET agent a peptide is complexed with one of the various positron emitting metal ions, such as ⁵¹Mn, ⁵²Fe, ⁶⁰Cu, ⁶⁸Ga, ⁷²As, ^{94m}Tc, ¹¹⁰In. Preferred metal radionuclides include ⁹⁰Y, ^{99m}Tc, ¹¹¹In, ⁴⁷Sc, ⁶⁷Ga, ⁵¹Cr, ^{177m}Sn, ⁶⁷Cu, ¹⁶⁷Tm, ⁹⁷Ru, ¹⁸⁸Re, ¹⁷⁷Lu, ¹⁹⁹Au, ²⁰³Pb, and ¹⁴¹Ce. ^{99m}Tc is preferred because of its low cost, av imaging properties, and high specific activity. The nuclear and radioactive properties of Tc-99m make this isotope an ideal scintigraphic imaging agent. This isotope has a single photon energy of 140 keV and a radioactive half-life of about 6 hours, and is readily available from a ⁹⁹Mo-^{99m}Tc generator. The radioactive metals may be chelated by, for example, linear, macrocyclic, terpyridine, and N₃S, N₂S₂, or N₄ chelants (see also, U.S. Pat. No. 5,367,080, U.S. Pat. No. 5,364,613, U.S. Pat. No. 5,021,556, U.S. Pat. No. 5,075,099, U.S. Pat. No. 5,886,142), and other chelators known in the art including, but not limited to, DTPA, EDTA, DOTA, TETA, and bisamino bisthiol (BAT) chelators (see also U.S. Pat. No. 5,720,934). The chelates may be covalently linked directly to the fibrin binding moiety or linked to the fibrin binding moiety via a linker, as described previously, and then directly labeled with the radioactive metal of choice (see, WO 98/52618, U.S. Pat. No. 5,879,658, and U.S. Pat. No. 5,849,261).

In forming a complex of radioactive technetium with the reagents of this invention, the technetium complex, preferably a salt of Tc-99m pertechnetate, is reacted with the reagent in the presence of a reducing agent. Preferred reducing agents are dithionite, stannous and ferrous ions; the most preferred reducing agent is stannous chloride. Means for preparing such complexes are conveniently provided in a kit form comprising a sealed vial containing a predetermined quantity of a reagent of the invention to be labeled and a sufficient amount of reducing agent to label the reagent with Tc-99m. Alternatively, the complex may be formed by reacting a peptide of this invention conjugated with an appropriate chelator with a pre-formed labile complex of technetium and another compound known as a transfer ligand. This process is known as ligand exchange and is well known to those skilled in the art. The labile complex may be formed using such transfer ligands as tartrate, citrate, gluconate or mannitol, for example. Among the Tc-99m pertechnetate salts useful with the present invention are included the alkali metal salts such as the sodium salt, or ammonium salts or lower alkyl ammonium salts.

Radioactively-labeled scintigraphic imaging agents provided by the present invention are provided having a suitable amount of radioactivity. In forming Tc-99m radioactive complexes, it is generally preferred to form radioactive complexes in solutions containing radioactivity at concentrations of from about 0.01 millicurie (mCi) to 100 mCi per mL.

Generally, the unit dose to be administered has a radioactivity of about 0.01 mCi to about 100 mCi, preferably 1 mCi to 20 mCi. The solution to be injected at unit dosage is from about 0.01 nL to about 10 mL.

Typical doses of a radionuclide-labeled fibrin binding imaging agents according to the invention provide 10-20 mCi. After injection of the fibrin-specific radionuclide imaging agent into the patient, a gamma camera calibrated for the gamma ray energy of the nuclide incorporated in the imaging agent is used to image areas of uptake of the agent and quantify the amount of radioactivity present in the clot. Imaging of the thrombus in vivo can take place in a matter of a few minutes. However, imaging can take place, if desired, in hours or even longer, after the radiolabeled peptide is injected into a patient. In most instances, a sufficient amount of the administered dose will accumulate in the area to be imaged within about 0.1 of an hour to permit the taking of scintiphotos.

Therapeutic Applications

The fibrin binding polypeptides of the present invention can be used to improve the activity of thrombolytic agents against clots by providing or improving their affinity for fibrin. In this aspect of the invention, hybrid thrombolytic agents are provided by conjugating a fibrin binding polypeptide according to the invention with a thrombolytic agent. The fibrin binding polypeptide portion of the conjugate causes the thrombolytic to “home” to the sites of fibrin clots, and to improve the affinity of the conjugate for the clots, so that the thrombolytic activity of the conjugate is more localized and concentrated at the sites of clots. Such conjugates will be useful in treating thrombus associated diseases, especially acute myocardial infarction, in humans and animals, which method comprises administering to a human or animal in need thereof an effective amount of a fibrin binding moiety according to the invention conjugated with a thrombolytic agent. The invention also provides the use of such conjugates in the manufacture of a medicament for the treatment of thrombus associated diseases in humans and animals. Suitable thrombolytic agents for use in this aspect of the invention include fibrinolytic enzymes, including plasminogen activators. The term plasminogen activator includes but is not limited to streptokinase, human tissue plasminogen activator (tPA) and urokinase (both single and two-chain forms). Such enzymes are obtained from natural sources or tissues or by recombinant production, discussed above. Other suitable thrombolytic agents include fibrinolytically active hybrid proteins (see, e.g., EP-A-155 387) which comprise one chain of a 2-chain protease linked to a chain of a different 2-chain protease, at least one of the chains in the hybrid protein being derived from a fibrinolytically active protease; thrombolytic protein conjugates (see, e.g., EP-A-152 736), such as urokinase linked to reversibly blocked plasmin; derivatives of fibrinolytic enzymes in which the catalytic site on the enzyme which is responsible for fibrinolytic activity is blocked by a human protein attached thereto by way of a reversible linking group, for example urokinase reversibly linked to the active center of human plasmin; genetically engineered derivatives including muteins of naturally occurring plasminogen activators; hybrid molecules (see, e.g., EP-A-297 882); reversibly blocked in vivo fibrinolytic enzymes, such as a binary complex between streptokinase and plasminogen, most preferably a p-anisoyl streptokinase/plasminogen complex without internal bond cleavage (anistreplase, described in U.S. Pat. No. 4,808,405); and the like.

The thrombolytic agents and the fibrin binding moieties can be linked or fused in known ways, using the same type of linkers discussed above with respect to constructing MRI contrast agents. Preferred linkers will be substituted or unsubstituted alkyl chains, amino acid chains, polyethylene glycol chains, and other simple polymeric linkers known in the art. More preferably, if the thrombolytic agent is itself a protein, for which the encoding DNA sequence is known, the thrombolytic protein and fibrin binding polypeptide may be coexpressed from the same synthetic gene, created using recombinant DNA techniques, as described above. The coding sequence for the fibrin binding polypeptide may be fused in frame with that of the thrombolytic protein, such that the peptide is expressed at the amino- or carboxy-terminus of the thrombolytic protein, or at a place between the termini, if it is known that such placement would not destroy the required biological function of either the thrombolytic protein or fibrin binding polypeptide. A particular advantage of this general approach is that concatamerization of multiple, tandemly arranged fibrin binding polypeptides is possible, thereby increasing the number and concentration of fibrin binding sites associated with each thrombolytic protein. In this manner fibrin binding avidity is increased which would be expected to improve the efficacy of the recombinant therapeutic protein.

In the above treatment method, the compounds may be administered by any convenient route customary for thrombolytic agents, for example by infusion or bolus injection. In a preferred embodiment, the composition may be formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anaesthetic such as lignocaine to ease pain at the site of the injection. Generally, the ingredients will be supplied either separately or mixed together in unit dosage form, for example, as a dry lyophilised powder or water free concentrate in a hermetically sealed container such as an ampoule or sachette indicating the quantity of active agent in activity units. Where the composition is to be administered by infusion, it can be dispensed with an infusion bottle containing sterile pharmaceutical grade “water for injection” or saline. Where the composition is to be administered by injection, an ampoule of sterile water for injection or saline may be provided so that the ingredients may be mixed prior to administration.

The quantity of material administered will depend on the seriousness of the thromboembolic condition and position and size of the clot. The precise dose to be employed and mode of administration must per force in view of the nature of the complaint be decided according to the circumstances by the physician supervising treatment. In general, dosages of the fibrin binder/thrombolytic agent conjugate will follow the dosages that are routine for the thrombolytic agent alone, although the improved affinity for fibrin added by the fibrin binder component may allow a decrease in the standard thrombolytic dosage. Particular thrombolytics contemplated for use in this therapy (with examples of dose and method of administration) are as follows:
streptokinase 1.0-3.0 megaunits over 30 minutes to 3 hours
anistreplase 30 units; 2-5 minute injection
tPA (wild-type) 50-150 mg; infusion over up to 6 hours
two-chain urokinase 40-100 mg; infusion over up to 6 hours
single-chain urokinase (3-12 megaunits) 30-100 mg; infusion over up to 5 hours
hybrid plasminogen 20-100 mg; injection or infusion activators and derivatives
muteins of plasminogen 10-100 mg; injection or infusion activators

In preferred features, the fibrin binding moiety is linked to the thrombolytic agent with a linker encompassing an enzymatic cleavage site, e.g., an enzymatic cleavage site normally cleaved by enzymes in the coagulation cascade, such as Factor Xa, thrombin, or plasmin cleavage sites, etc. The thrombolytic agent preferably would not be activated until it is cleaved from the fibrin binding moiety at the site of the clot. Since cleavage of the thrombolytic agent would occur at the site of the clot, the risk of unwanted bleeding events at sites distant from the clot would be minimized.

Alternatively, a therapeutic thrombolytic can be loaded into an ultrasound vesicle that has been derivatized on its surface with the fibrin binding moieties of the present invention. The vesicle may also be filled with an ultrasound efficient gas, such as, but not limited to, perfluoropropane or perfluorobutane. Once the fibrin-specific vesicle has homed to the site of a thrombus, as monitored by ultrasound, the frequency and energy of the ultrasound waves administered can be altered to result in a controlled release of the thrombolytic at the site of the thrombus (see, e.g., WO 93/25241).

Pharmaceutical Applications

Whether the fibrin binding moieties are to be used in patients for detection and diagnosis or to facilitate the therapeutic degradation of thrombi, such uses require that they be treated as pharmaceutical agents. Pharmaceutical compositions of this invention comprise any of the compounds of the present invention, and pharmaceutically acceptable salts thereof, with any pharmaceutically acceptable ingredient, excipient, carrier, adjuvant or vehicle.

Pharmaceutical compositions of this invention can be administered to mammals including humans in a manner similar to other diagnostic or therapeutic agents. The dosage to be administered, and the mode of administration will depend on a variety of factors including age, weight, sex, condition of the patient, and genetic factors, and will ultimately be decided by the attending physician or veterinarian. In general, dosage required for diagnostic sensitivity or therapeutic efficacy will range from about 0.001 to 50,000 μg/kg, more usually 0.01 to 25.0 μg/kg of host body mass.

Pharmaceutically acceptable salts of the compounds of this invention include, for example, those derived from pharmaceutically acceptable inorganic and organic acids and bases. Examples of suitable acids include hydrochloric, hydrobromic, sulfuric, nitric, perchloric, flimaric, maleic, malic, pamoic, phosphoric, glycolic, lactic, salicylic, succinic, toluene-p-sulfonic, tartaric, acetic, citric, methanesulfonic, formic, benzoic, malonic, naphthalene-2-sulfonic, tannic, carboxymethyl cellulose, polylactic, polyglycolic, and benzenesulfonic acids. Other acids, such as oxalic, while not in themselves pharmaceutically acceptable, may be employed in the preparation of salts useful as intermediates in obtaining the compounds of the invention and their pharmaceutically acceptable acid addition salts. Salts derived from appropriate bases include alkali metal (e.g., sodium), alkaline earth metal (e.g., magnesium), ammonium and N—(C₁₋₄ alkyl)₄⁺ salts.

This invention also envisions the “quatemization” of any basic nitrogen-containing groups of the compounds disclosed herein. The basic nitrogen can be quatemized with any agents known to those of ordinary skill in the art including, for example, lower alkyl halides, such as methyl, ethyl, propyl and butyl chloride, bromides and iodides; dialkyl sulfates including dimethyl, diethyl, dibutyl and diamyl sulfates; long chain halides such as decyl, lauryl, myristyl and stearyl chlorides, bromides and iodides; and aralkyl halides including benzyl and phenethyl bromides. Water or oil-soluble or dispersible products may be obtained by such quaternization.

It should be understood that the compounds of this invention may be modified by appropriate functionalities to enhance selective biological properties. Such modifications are known in the art and include those which increase biological penetration into a given biological system (e.g., blood, lymphatic system, central nervous system), increase oral availability, increase solubility to allow administration by injection, alter metabolism and alter rate of excretion. In addition, the compounds may be altered to pro-drug form such that the desired compound is created in the body of the patient as the result of the action of metabolic or other biochemical processes on the pro-drug. Such pro-drug forms typically demonstrate little or no activity in in vitro assays. Some examples of pro-drug forms include ketal, acetal, oxime, and hydrazone forms of compounds which contain ketone or aldehyde groups. Other examples of pro-drug forms include the hemi-ketal, hemi-acetal, acyloxy ketal, acyloxy acetal, ketal, and acetal forms.

Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The pharmaceutical compositions of this invention may be administered by a variety of routes or modes. These include, but not limited, to oral, intratracheal, sublingual, pulmonary, topical, rectal, nasal, buccal, vaginal, parenteral, or via an implanted reservoir. Implanted reservoirs may function by mechanical, osmotic, or other means. The term parenteral as used herein includes intraperitoneal, paravertebral, periarticular, periostal, subcutaneous, intracutaneous, intravenous, intra-arterial, intramuscular, intra-articular, intrasynovial, intrasternal, intrathecal, intralesional and intracranial injection or infusion techniques.

Such compositions are preferably formulated for parenteral administration, and most preferably for intravenous or intra-arterial administation. Generally, and particularly when administration is intravenous or intra-arterial, pharmaceutical compositions may be given as a bolus, as two or more doses separated in time, or as a constant or non-linear flow infusion.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant such as those described in Pharmacoplia Halselica.

The pharmaceutical compositions of this invention may be orally administered in any orally acceptable dosage form including, but not limited to, capsules, tablets, caplets, pills, aqueous or oleaginous suspensions and solutions, syrups, or elixirs.

Pharmaceutical compositions of the invention may be packaged in a variety of ways appropriate to the dosage form and mode of administration. These include but are not limited to vials, bottles, cans, packets, ampoules, cartons, flexible containers, inhalers, and nebulizers. Such compositions may be packaged for single or multiple administations from the same container. Kits of one or more doses may be provided containing both the composition in dry powder or lyophilized form, as well an appropriate diluent, which are to be combined shortly before administration. The pharmaceutical composition may also be packaged in single use prefilled syringes, or in cartridges for autoinjectors and needleless jet injectors.

Multiuse packaging may require the addition of antimicrobial agents such as phenol, benzyl alcohol, meta-cresol, methyl paraben, propyl paraben, benzalconium chloride, and benzethonium chloride, at concentrations that will prevent the growh of bacteria, fungi, and the like, but be non-toxic when administered to a patient.

Consistent with good manufacturing practices which are in current use in the pharmaceutical industry and which are well known to the skilled practioner, all components contacting or comprising the pharmaceutical agent must be sterile and periodically tested for sterility in accordance with industry norms. Methods for sterilization include ultrafiltration, autoclaving, dry and wet heating, exposure to gases such as ethylene oxide, exposure to liquids, such as oxidizing agents, including sodium hypochlorite (bleach), exposure to high energy electromagnetic radiation, such as ultraviolet light, x-rays or gamma rays, and exposure to ionizing radiation. Choice of method of sterilization will be made by the skilled practioner with the goal of effecting the most efficient sterilization that does not significantly alter the biological function of the pharmaceutical agent in question. Regarding the fibrin binding moieties of the present invention, ultrafiltration is the preferred method of sterilization for pharmaceutical compositions that are aqueous solutions or suspensions.

Details concerning dosages, dosage forms, modes of administration, composition and the like are further discussed in a standard pharmaceutical text, such as Remington's Pharmaceutical Sciences, 18th ed., Alfonso R. Gennaro, ed. (Mack Publishing Co., Easton, Pa. 1990), which is hereby incorporated by reference.

Isolation of fibrin binding moieties in accordance with this invention will be further illustrated in the following examples. The specific parameters included in the following examples are intended to illustrate the practice of the invention, and they are not presented to in any way limit the scope of the invention.

### EXAMPLE 1

### Preparation of a Fibrin Target for Library Screening

For screening libraries to isolate binding moieties for fibrin, two fibrin targets, i.e., synthetic fibrin clots and then a soluble fibrin fragment, DD(E), were prepared. To prepare fibrin for screening, dilute fibrin clots were formed in the wells of a 96-well plate, dried down to a thin layer, and then rehydrated prior to library screening. In a typical procedure, a 0.15 mg/ml fibrinogen solution was prepared in TBS buffer (50 mM Tris, 150 mM NaCl, pH 7.4). A solution containing 2 U/ml thrombin, 10 mM CaCl₂, and 5 mM ε-aminocaproic acid in TBS was prepared. The fibrinogen solution and thrombin solution were mixed 1:1 in the wells of a 96-well plate, aliquoting 25 μL of each solution in each well (total volume=50 μL). The plates were evaporated to dryness by incubating them at 37° C. overnight. Just before a phage library was added to the dried fibrin target, the fibrin wells were washed three times for 10 minutes with phage blocking buffer (TBS containing 2 mM CaCl₂, 0.1% Tween-20, and 0.1% human serum albumin (HSA)).

The soluble fibrin-derived polypeptide DD(E) was prepared following a modification of a published method (Moskoitz and Budzynksi, Biochemistry, 33: 12937-12944 (1994) and references therein). Fibrinogen containing a Factor XIII trace impurity (1 g, Grade L; purchased from American Diagnostica) was dissolved in TBS buffer and dialyzed overnight against TBS containing 5 mM citrate. The fibrinogen concentration was adjusted to 3.0 mg/ml and CaCl₂ was added to a concentration of 10 mM. Clotting of the fibrinogen was initiated by the addition of thrombin to 0.5 U/ml and the clot was incubated for 3 hours at 37° C. The clot was cut up with a spatula to release water and to concentrate the clot. The clot pieces were washed twice with TBS.Ca buffer (TBS containing 2 mM CaCl₂) and were centrifuged at 4,000×g to compact the clots between washes. The clot material was resuspended in 250 ml TBS containing 25 mM CaCl₂ and 2 K.I.U. plasmin per mg fibrin. The clots were digested overnight at 20° C. Undigested clot was removed by pipette, and the supernatant was shaken with 10 ml of Lysine Sepharose (Pharmacia) for 30 minutes and filtered to remove the resin. Aprotinin was added to the filtrate to a concentration of 500 U/μL. Ammonium Sulfate was added to 30% saturation and the precipitated protein was removed by centrifugation. More ammonium sulfate was added to the supernatant to a final concentration of 50% saturation, and the precipitated protein was concentrated by centrifugation. The pellets, containing DD(E), were resuspended in a small volume of buffer (50 mM Tris, 150 mM NaCl, 2 mM CaCl₂) (<10 ml) and chromatographed on a Sephacryl S200 (Pharmacia) size exclusion column (5×100 cm) in the same buffer. Fractions of protein eluted from the column were assayed by SDS-PAGE for DD(E). DD(E) contains subunits of 55 kD (Fragment E) and 190 kD (Fragment DD).

To prepare the DD(E) as a target for library screening, the complex was first biotinylated. The buffer was changed to 50 mM sodium phosphate and reacted with 10 equivalents of sulfo-NHS-biotin (Pierce Chemical Co.), an amino-functional compound that adds biotin moiety to amine-reactive sites. The biotin binding protein streptavidin was then immobilized by passive binding to the bottom of the wells of polystyrene 96-well microtiter plates, and the biotinylated DD(E) was added to these plates. Roughly 100 pmol of DD(E) were immobilized per well. Excess DD(E) was washed off the plates, which were then incubated with buffer containing 50 mM Tris, 150 mM NaCl, 2 mM CaCl₂, 0.05% Tween-20, and 0.1% human serum albumin to block sites against nonspecific binding.

### EXAMPLE 2

### Screening of Phage Display Libraries

The TN7 phage library displaying variegated exogenous single-loop peptides based on a polypeptide template having the structure Xaa-Xaa-Cys-Xaa-Xaa-Xaa-Xaa-Xaa-Cys-Xaa-Xaa (SEQ ID NO: 30) (5×10⁹ peptide diversity displayed on M13 phage) was diluted into 100 μL of binding buffer (50 mM Tris, 150 mM NaCl, 2 mM CaCl₂, 0.05% Tween-20).

Before selecting phage that bound to the fibrin or DD(E) targets, at the beginning of each screening round, the libraries were depleted of fibrinogen binders: Fibrinogen was biotinylated by the same method employed for DD(E) biotinylation, and immobilized on magnetic beads. The beads were aliquoted into five tubes. The phage library was incubated with the beads in the first tube for 10 minutes, the beads were pelleted with a magnet, and the supernatant, now at least partially depleted of fibrinogen binding phage, was transferred to a second tube. This process was repeated over the five tubes, and after the last depletion, the library was introduced to the microtiter plates containing the immobilized DD(E) target, prepared as above. After a 2-hour incubation with the target to allow binding of phage to DD(E), the wells of the plate were washed extensively (15 times) to remove unbound or weakly bound phage. Bound phage were recovered by eluting the phage from the target in pH 2.0 citrate buffer (10 mM citrate, 150 mM NaCl). The recovered phage were propagated and prepared for use in the succeeding round of selection. In all, five rounds of depletion and selection were conducted. After each round, the phage eluted were counted to determine if the amount of phage recovered (as a percent of the input) increased, an indication that the screening process was converging on a small family of sequences.

### EXAMPLE 3

### Analysis of Individual Isolates

After five rounds of selection, the eluted phage were propagated and a portion plated to isolate phage plaques arising from individual clones. Ninety such clones were selected randomly, propagated, and tested individually for binding to fibrin in a dried fibrin plate assay. Dried fibrin plates were prepared as described above for the library screening. Phage samples (˜10⁹ phage each) were incubated in the dried fibrin plate wells in binding buffer (50 mM Tris, 150 mM NaCl, 2 mM CaCl₂, 0.05% Tween-20) containing 0.1% HSA. After 1 hour, the plates were washed 5 times with binding buffer. Anti-M13 antibody conjugated to horseradish peroxidase (Pharmacia) was added at 1/5000 dilution in binding buffer to the wells and incubated with the fibrin for 1 hour. The wells were again washed 5 times with binding buffer and the presence of the antibody/phage/fibrin complex was measured with HRP calorimetric reagents (3,3′,5,5′-tetrarnethylbenzidine (TMB) and H₂O₂). A high absorbance at 595 nm (due to oxidized TMB) was indicative of a tight phage/fibrin interaction, and phage clones corresponding to those wells were identified as bearing fibrin-binding moieties.

These fibrin-binding positive clones were subjected to several secondary ELISA assays. These assays followed a similar protocol to that detailed above for the dried fibrin ELISA assay, the only variations being the method of target immobilization and the omission of HSA from binding and wash buffers. A screen against DD(E) acted as a further confirmation of fibrin binding activity. An ELISA screen against fibrinogen (immobilized to the plate by the biotin/streptavidin protocol used for DD(E)) was a check against fibrinogen binding and confirmed that the negative selection procedure detailed in Example 2 had been effective. Finally, ELISAs to assay binding to immobilized HSA (passively bound to the polystyrene plate) and a target-free microtiter plate were controls to eliminate phage that bound promiscuously or nonspecifically.

The amino acid sequences of the phage-displayed polypeptides from the ELISA positive clones (those positive for fibrin, but negative for fibrinogen, HSA and the polystyrene plate) were deduced by DNA sequencing. The amino acid sequence data from these phage isolates were sorted according to the degree of similarity and response in the fibrin ELISA assay. The results of the screen from the TN7 library are set forth in Table 1.

TABLE 1

Amino acid sequences of fibrin-binding
polypeptides identified from the TN7 library

| | | | |
|---|---|---|---|
| TN7 | | relative | SEQ |
| isolate | sequence | binding | ID NO: |

| | | | |
|---|---|---|---|
| 1 | R P C D Y Y G T C F D | + + + | 8 |
| 2 | L S C D Y Y G T C L R | + + + | 13 |
| 3 | L P C D Y Y G T C L D | + + + | 9 |
| 4 | D P C S Y Y G T C L H | + + + | 11 |
| 5 | L P C S Y Y G T C L H | + + + | 12 |
| 6 | F A C H Y Y G T C L H | + + + | 7 |
| 7 | L A C H Y Y G T C L H | + + + | 14 |
| 8 | D G C H Y Y G T C L H | + + + | 15 |
| 9 | R S C N Y Y G T C L H | + + + | 5 |
| 10 | R P C N Y Y G T C L H | + + + | 16 |
| 11 | H D C Q Y Y G T C L H | + | 6 |
| 12 | F S C W Y S L H C H R | + | 10 |

### EXAMPLE 4

### Binding Studies with Polypeptides Prepared Using Solid Phase Synthesis

The polypeptide sequences binding to fibrin target define a cysteine-bracketed fibrin binding loop of seven amino acids (including the cysteines), viz., Cys-Xaa-Tyr-Tyr-Gly-Thr-Cys, where Xaa is Asn, Asp, Gln, His, Ser or Trp (SEQ ID NO:3), which forms a stable binding site for fibrin and not fibrinogen. It is also clear from this family that a new fibrin binding peptide has been identified, i.e., Tyr-Tyr-Gly-Thr (SEQ ID NO: 4) which will be helpful in the design of additional fibrin binding moieties.

Even though a fibrin binding loop formed by a disulfide link between the cysteine residues of the polypeptides isolated from the TN7 library presents a conformationally stable structure, the binding loop would still possess considerable conformational freedom. In order to investigate the effects of reducing the complexity due to conformational flexibility of the longer polypeptides and to investigate those residues of the polypeptide that are most important for binding and activity at the active site of fibrin, a series of polypeptides was prepared based on the structure X₁—X₂-Cys-X₄-Tyr-Tyr-Gly-Thr-Cys-X₁₀—X₁₁ (SEQ ID NO: 31), where X₁, X₂, X₄, X₁₀, and X₁₁ are the residues specified in Table 2, below.

TABLE 2
Amino acid sequence of fibrin binder homologues

| | | | | | | | | | | | |
|---|---|---|---|---|---|---|---|---|---|---|---|
| | | | | | | | | | | | SEQ. ID |
| X₁ | X₂ | C | X₄ | Y | Y | G | T | C | X₁₀ | X₁₁ | NO: |

| | | | | | | | | | | | |
|---|---|---|---|---|---|---|---|---|---|---|---|
| L | P | C | D | Y | Y | G | T | C | L | D | 9 |
| R | P | C | D | Y | Y | G | T | C | F | D | 8 |
| F | A | C | H | Y | Y | G | T | C | L | H | 7 |
| R | P | C | N | Y | Y | G | T | C | L | H | 16 |
| L | P | C | S | Y | Y | G | T | C | L | H | 12 |

Investigation of such polypeptides allowed a systematic alteration of the conformational space of the binding loop and a sequential variation of the spatial orientation of the residues by a fixed increment. (See examples, infra.) The binding of the 11-mer polypeptides was surprisingly similar to the parent phage-presented peptides from which they were derived. The dissociation constants with respect to fibrin were determined using the following method:

A fibrinogen solution was prepared at 10 mg/ml (or at twice the concentration of fibrin desired) in TBS buffer (50 mM Tris, 150 mM NaCl, pH 7.4). The fibrinogen solution typically contained ˜17 mM citrate. Subsequently, a solution was prepared containing 2 U/ml thrombin, 20 mM CaCl₂, and 5 mM ε-aminocaproic acid in TBS. The fibrinogen solution and thrombin solution were mixed 1:1 in the wells of a 96-well plate, aliquoting 50 μL of each solution in each well (total volume=100 μL). The plates were evaporated to dryness overnight at 37° C. The polypeptide to be tested, dissolved in water, was added to each well at concentrations between 1-200 μM. A typical binding assay contained 24 points at concentrations of 2, 4, 6, 8, 10, 12, 14, 17, 20, 23, 26, 30, 35, 40, 45, 50, 60, 70, 80, 100, 125, 150, 175, and 200 μM. The plate containing the peptide and (rehydrated) dried fibrin was covered and incubated at 37° C. on a shaker table for 2 hours.

The supernatant in each well was removed by pipette and the concentration of the polypeptide was measured by mass spectrometry. The ion current detected at the mass of the peptide was monitored after injection of a sample into the mass spectrometer. The area-under-the-peak was quantitated and compared to standards of known concentration. The concentration of peptide in the supernatant is equal to the concentration of free peptide. The concentration of bound peptide was determined by subtracting the concentration of free peptide from the total (starting) concentration. A plot of [Bound Peptide] vs. [Free Peptide] was used to determine the K_{d} and the concentration of bound peptide at saturation. The curve was fit to the equation:
[Bound]=N×[Free]/(K_{d}+[Free])
where [Bound] is the concentration of bound polypeptide, [Free] is the concentration of free polypeptide, K_{d} is the dissociation constant (equal to the reciprocal of Kₐ) and N is the concentration of binding sites. The number of binding sites per fibrin molecule was equal to the lo concentration of binding sites determined by the [Bound Polypeptide] vs. [Free Polypeptide] plot, divided by the concentration of fibrin (15 μM) used in the assay.

The dissociation constants for the synthetic fibrin binding polypeptides is set forth in Table 3, below.

TABLE 3

Dissociation constants (K_{d})
for fibrin binding polypeptides

| | | | |
|---|---|---|---|
| | Amino | K_{d} | K_{d} |
| Polypeptide | Acid Sequence | dried fibrin | DD(E) |

| | | | | |
|---|---|---|---|---|
| SEQ ID NO: 9 | LPCDYYGTCLD | 2.7 | μM | 2.6 μM |
| SEQ ID NO: 8 | RPCDYYGTCFD | 6.8 | μM | 3.4 μM |
| SEQ ID NO: 7 | FACHYYGTCLH | 23 | μM | 7.3 μM |
| SEQ ID NO: 16 | RPCNYYGTCLH | 8.5 | μM | 4.0 μM |
| SEQ ID NO: 12 | LPCSYYGTCLH | 13 | μM | 9.8 μM |

Systematic deletions from the C and the N terminus of one of the polypeptides were carried out to identify the minimum binding fragment of the larger polypeptide. As before, the C terminus was capped as an amide and the N-terminus was free. Binding affinity for fibrin expressed as a dissociation constant (K_{d}) was determined using the method described above. The additional truncated polypeptides are shown in Table 4, below:

TABLE 4
Truncated fibrin binding peptides

| | | |
|---|---|---|
| | K_{d} | SEQ ID |
| Amino Acid Sequence | DD(E) | NO: |

| | | | |
|---|---|---|---|
| PCDYYGTCL | 50 | μM | 32 |
| CDYYGTCL | 22 | μM | 33 |
| CDYYGTC | 38 | μM | 34 |
| DYYGT | >100 | μM | 35 |

The results of the foregoing experiments confirm that the YYGT peptide is a new binding fragment recognized by an epitope of fibrin formed in the DD(E) region. Polypeptides containing this fibrin binding fragment would thus be expected to have some binding affinity for fibrin.

### EXAMPLE 5

### Fibrin Binding Polypeptides Obtained by Screening Additional Libraries

Following similar procedures to those described above for isolation of fibrin binding polypeptides from the TN7 phage display library, additional fibrin binding polypeptides were isolated by screening two additional phage display libraries designated TN10-9 and TN6-6. The TN10-9 phage library displayed variegated exogenous single-loop peptides based on a template of the structure Xaa-Xaa-Xaa-Cys-Xaa-Xaa-Xaa-Xaa-Xaa-Xaa-Xaa-Xaa-Cys-Xaa-Xaa-Xaa (SEQ ID NO: 36); the TN6-6 phage library displayed variegated exogenous single-loop peptides based on a template of the structure Xaa-Xaa-Xaa-Cys-Xaa-Xaa-Xaa-Xaa-Cys-Xaa-Xaa-Xaa (SEQ ID NO: 37).

Phage isolates binding to fibrin target (but not irrelevant controls) from these libraries displayed exogenous polypeptides having the amino acid sequences set forth in Table 5. Dissociation constants were determined against DD(E) as described above.

TABLE 5

Amino acid
sequences of fibrin-binding polypeptides
identified from the TN10-9 and TN6-6 libraries

| | | | |
|---|---|---|---|
| | | | SEQ |
| | | K_{d} | ID |
| Library | Amino Acid Sequence | DD(E) | NO: |

| | | | | |
|---|---|---|---|---|
| TN10-9 | N H G C Y N S Y G V P Y C D Y S | 2.7 | μM | 18 |
| TN10-9 | R F L C Y D S Y Y H W W C S H H | ˜10 | μM | 19 |
| TN6-6 | W F H C P Y D L C H I L | 1.4 | μM | 21 |
| TN6-6 | Q W E C P Y G L C W I Q | 0.74 | μM | 22 |

Synthetic peptides prepared according to amino acid sequence data from the TN6-6 isolates by solid phase synthesis, as described above, were also tested for binding to a DD(E) target. The data are shown in Table 6, below. Binding affinity was seen to be comparable to that of the phage isolates.

TABLE 6

Dissociation constants (K_{d})
for fibrin binding polypeptides

| | | K_{d} |
|---|---|---|
| Polypeptide | Amino Acid Sequence | DD(E) |
| SEQ ID NO: 21 | W F H C P Y D L C H I L | 2.63 μM |
| SEQ ID NO: 22 | Q W E C P Y G L C W I Q | 2.96 μM |

### EXAMPLE 6

### Further Modification of a Fibrin Binding Loop

Peptide analogs were prepared by substituting amino acids and incorporating non-natural amino acids at the cysteine positions of the TN7 fibrin binding loop, to increase or further restrict the conformational freedom of the polypeptides. The two cysteines were first replaced with serines (isoelectronic replacement) to give a linear peptide. The cysteine-to-serine replacement eliminates the possibility of forming a disulfide bond and would indicate if a cyclic structure contributes to binding. Serine-substituted linear polypeptides also provide a comparative tool to investigate binding affinity between a linear polypeptide vs. a cyclic structure. Second, in order to decrease the flexibility of the binding loop, penicillamine (β,β-dimethylcysteine, abbreviated “pen” in Table 7) was substituted for the two cysteine residues.

The linear peptides were synthesized by the solid-phase method with rink-amide AM resin at a substitution level of 0.8 mmou/g. Typically 0.3-0.6 g of resin was used for each synthesis. All amino acids were protected with N^{α}-(Fluorenyloxy)carbonyl (Fmoc). Side chain protecting groups were Arg(Pmc), Asp(OtBu), Cys(Acm), Glu(OtBu), His(Trt), Ser(OtBu), Thr(OtBu), Tyr(OtBu), Trp(Boc).

Each synthetic cycle consisted of an 18-minute deprotection with 25% piperidine in DMF, several wash steps with DMF (at least twice) and MeOH, followed by double coupling with preformed OBt esters (6 equivalents of the Fmoc amino acid) 1 hour with dicyclohexylcarbodiimide (DIC), and subsequently 0.5 hour with [2-(1H-benzotriazole-1-yl)-1,1,3,3,-tetrarnethyluronium hexafluorophosphate]/DIEA (diisopropylethylamine). All couplings were monitored by the qualitative ninhydrin test. After complete assembly and N-terminal Fmoc deprotection, and after washing the resin with DMF and MeOH, the peptide was cleaved from the resin. The peptide-resin was treated with a trufluoroacetic acid (TFA) cocktail comprising of (94% TFA, 2.5% TES (triethylsilane), 2.5% water and 1% anisole) and stirred for 2 hours at room temperature. The resin was filtered off and was washed several times with cold TFA. The combined pale yellow filtrate was then concentrated on a SAVANT SpeedVac rotary evaporator (Model SC250DDA, Savant Instruments, Holbrook N.Y.) to about 2% of the original volume. This concentrated filtrate was poured into excess cold ether and a white solid separated out. The solution was centrifuged and the pellet obtained was further triturated with ether. After centrifugation, the supernatant was discarded and the pellet was dissolved in 5% AcOH and was purified on a size-exclusion column using MeOH.

All linear peptide analogues were purified by C₁₈ or C₄ reverse-phase HPLC using a gradient of acetonitrile (0.1% TFA) and H₂O (0.1% TFA). The S-Acm protected cysteine peptide in TFA was treated with T1(CF₃COO)₃ (1.1 eq.) in the presence of anisole in an ice-bath for 60 min. The progress of the reaction was monitored by HPLC. At the end of the reaction the TFA was evaporated under a vacuum. The combined pale yellow filtrate was then concentrated on a SAVANT SpeedVac rotary evaporator system to about 2% of the original volume. This concentrated filtrate was poured into excess cold ether and a white solid separated out. The solution was centrifuged and the pellet obtained was further triturated with ether. After centrifugation, the supernatant was discarded and the pellet was dissolved in 5% AcOH and was purified on RP-HPLC using a gradient of ACN(0.1% TFA) and H₂O (0.1% TFA) to a white fluffy solid in overall yield of 12-15% based on the starting resin substitution level. The integrity of the purified product was confirmed by electrospray mass spectrometry.

The modified polypeptides were tested for fibrin affinity using the method described previously. The effect of the modifications on affinity are seen in Table 7.

TABLE 7

Fibrin binding peptides linearized and
restricted with di-penicillamine substitutions

| | K_{d} | K_{d} | SEQ ID |
|---|---|---|---|
| Amino Acid Sequence | dried fibrin | DD(E) | NO: |
| LPSDYYGTSLD | >100 μM | >100 μM | 38 |
| LPpenDYYGTpenLD | n.d.* | >100 μM | 39 |

*(n.d. = not determined)

### EXAMPLE 7

### Modification by D-amino Acid Substitutions

A fibrin binding polypeptide was modified by replacement of Gly in the Tyr-Tyr-Gly-Thr motif with a D-amino acid (D-Ala). Gly is not asymmetric, and it can adopt conformations available to either an L- or a D-amino acid. If in the binding conformation of the peptide, Gly behaves like a D-amino acid, then its substitution by a D-amino acid should lock the peptide in this conformation and enhance the binding of the peptide.

The chirality of the residues in the second and third (i+1 and i+2) positions of a β-turn affects the conformation of the turn. If both are of L-configuration, a Type I turn is preferred, whereas a Type II or Type II′ turn can result from an -LD or -DL pair. Glycine, which lacks an asymmetric center, can occupy either position. In order to establish the characteristic of the binding site for the isolates that have been identified (predicted to be Type I turn) the substitution of the Gly with D-Ala is proof positive because the substitution would lead to disruption of Type I turn and hence reduce the binding affinity. The results of this modification on fibrin affinity is seen in Table 8.

TABLE 8

D-alanine (dA) substitution
of fibrin binding peptides

| | | |
|---|---|---|
| | K_{d} | |
| Amino Acid Sequence | DD(E) | SEQ ID NO: |

| | | |
|---|---|---|
| LPCDYYdATCLD | 4.9 μM | 40 |

### EXAMPLE 8

### Modification by L-Alanine Substitutions

A systematic replacement of every residue within a fibrin binding polypeptide amino acid sequence with L-alanine was carried out. The cysteines were left intact so as to retain the ability to form the disulfide bond. The C terminus was capped as an amide and the N terminus was free. Effects on the fibrin affinity of the “alanine scan” are seen in Table 9.

TABLE 9
Alanine Scan of Fibrin Binding Polypeptides

| | | |
|---|---|---|
| | | K_{d} |
| | | dried fibrin |
| Polypeptide | Amino Acid Sequence | DD(E) |

| | | | | | |
|---|---|---|---|---|---|
| SEQ ID NO: 9 | LPCDYYGTCLD (parent) | 2.7 | μM | 2.6 | μM |
| SEQ ID NO: 41 | LACDYYGTCLD | 31 | μM | 2.9 | μM |
| SEQ ID NO: 42 | LPCAYYGTCLD | 23 | μM | 5.9 | μM |
| SEQ ID NO: 43 | LPCDAYGTCLD | 16 | μM | 4.5 | μM |
| SEQ ID NO: 44 | LPCDYAGTCLD | >100 | μM | >100 | μM |
| SEQ ID NO: 45 | LPCDYYATCLD | 41 | μM | 12.3 | μM |
| SEQ ID NO: 46 | LPCDYYGACLD | >100 | μM | >100 | μM |
| SEQ ID NO: 47 | LPCDYYGTCAD | 32 | μM | 10.1 | μM |

Based on the binding results of the alanine scan peptides and other peptides, further constraints were introduced at the Thr₈ and Tyr₆ positions (numbering from the N-terminus) which appeared to be key for binding.

Thr₈ was substituted with natural or non-natural amino acids that introduced —CH₂OH (serine), —CH(Me)₂ (valine), diaminopropionic acid (Dpr), and L-homoserine (Hse) side chains, in place of the —CHOH-Me side chain of threonine.

The Tyr₈ position was substituted to produce more hydrophobic analogs by introducing 1-naphthyl (Nal), biphenyl group (Bip), and also a cyclic phenylalanine analog, tetrahydroisoquinoline-3-carboxylic acid (Tic) for the —CH₂-Ph-OH side chain of tyrosine. The peptides were capped on the C-terminus as an amide and the N-terminus was free. The results of the modifications introduced to the isolates are set forth in Table 10.

TABLE 10
Effect of tyrosine and threonine substitutions

| | | |
|---|---|---|
| Polypeptide | Amino Acid Sequence | K_{d} DD(E) |

| | | | |
|---|---|---|---|
| SEQ ID NO: 9 | LPCDYYGTCLD (parent) | 2.6 | μM |
| SEQ ID NO: 48 | LPCDYYGSCLD | 26 | μM |
| SEQ ID NO: 49 | LPCDYYGDprCLD | >500 | μM |
| SEQ ID NO: 50 | LPCDYYGHseCLD | >500 | μM |
| SEQ ID NO: 51 | LPCDYYGVCLD | 28 | μM |
| SEQ ID NO: 52 | LPCDYFGTCLD | >500 | |
| SEQ ID NO: 53 | LPCDYNalGTCLD | 125 | |
| SEQ ID NO: 54 | LPCDNalGTCLD | >500 | |
| SEQ ID NO: 55 | LPCDYBipGTCLD | >500 | |
| SEQ ID NO: 56 | LPCDYTicGTCLD | >500 | |

### EXAMPLE 9

### Modification of TN6-6 Fibrin Binding Polypeptides

Using similar methods as in the previous examples, substituted and truncated homologues of the peptides identified from the TN6-6 library were prepared and tested for binding against a DD(E) target. The polypeptides and the binding data are shown in Table 11.

TABLE 11
Modified TN6-6 Polypeptides

| | | K_{d} |
|---|---|---|
| Polypeptide | Amino Acid Sequence | DD(E) |
| SEQ ID NO: 21 | WFHCPYDLCHIL (parent) | 2.63 μM |
| SEQ ID NO: 57 | GFHCPYDLCHIL | 6.70 μM |
| SEQ ID NO: 58 | FHCPYDLCHIL | 0.41 μM |
| SEQ ID NO: 59 | HCPYDLCHIL | 3.80 μM |
| SEQ ID NO: 60 | FHCPYDLCHI | 2.00 μM |
| SEQ ID NO: 22 | QWECPYGLCWIQ (parent) | 2.96 μM |
| SEQ ID NO: 61 | WECPYGLCWIQ | 0.94 μM |
| SEQ ID NO: 62 | ECPYGLCWIQ | 2.23 μM |
| SEQ ID NO: 63 | WECPYGLCWI | 3.87 μM |

In addition, a competitive DD(E) binding assay was performed using fluoresceinated peptides corresponding to polypeptides SEQ ID NOs: 9, 18, 19 and 22, described above. The competitive assay showed that the TN7 polypeptides competed with the TN10-9 polypeptide but did not compete for DD(E) fibrin binding with the peptides of the TN6-6 library. These results indicate that the TN6-6 polypeptides recognize a different site on fibrin than the polypeptides identified from the TN7 and the TN10-9 libraries.

### EXAMPLE 10

### Cyclization of Fibrin Binding Polypeptides

Cyclization by forming an amide bond between the N-terminal amino group and the C-terminal carboxyl group represents an alternative for binding loop formation, e.g., instead of having disulfide formation between the cysteine positions. Also, some of the side chains lend themselves to cyclization via reaction of amino-functional side chains, such as with lysine or diaminopropanoic acid and the carboxylic acid side chain of aspartic acid. Such head-to-tail (I, II) and side-chain-to-side-chain (III, IV) cyclic peptides bearing a central Tyr-Tyr-Gly-Thr moiety were prepared, having the structures shown in Table 12, below:

TABLE 12

| | |
|---|---|
| (I) | 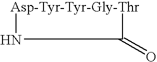 |
| (II) | 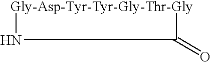 |
| (III) | 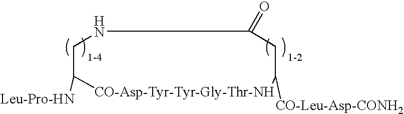 |
| (IV) | 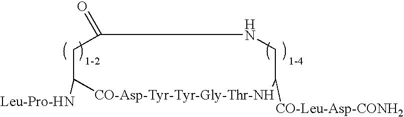 |

The dissociation constants (K_{d}) against the fibrin-derived peptide target, DD(E), were determined for the cyclic peptide of structure (II) (K_{d}>500) and a cyclic peptide according to structure (III) (K_{d}=179). These results indicate that adjustment of the cyclic structure is necessary to correctly configure the Tyr-Tyr-Gly-Thr moiety for high affinity fibrin binding.

### EXAMPLE 11

### Attachment of Chelates on the Isolates

The synthesis of a fibrin binder peptide-DTPA conjugate was performed by synthesizing N,N-bis[2-[bis[2-(1,1-dimethylethoxy)-2-oxoethyl]-amino]ethyl]-L-aspartic acid 1-(1,1-dimethylethyl) ester or in short, DTPA-Asp(β-COOH)—O^{t}Bu starting from L-aspartic acid-4-benzyl ester. Bisalkylation of protected L-aspartic acid with tert-butyl N-(2-bromoethyl)iminodiacetate, followed by catalytic hydrogenolysis of the gamma-benzyl ester affords DTPA-Asp(β-COOH)—O^{t}Bu. The DTPA-Asp(β-COOH)—O^{t}Bu was coupled to the N-terminus of a resin-bound synthetic fibrin binder peptide, Leu-Pro-Cys-Asp-Tyr-Tyr-Gly-Thr-Cys-Leu-Asp (SEQ ID NO:9), designated Pep1 and illustrated below, 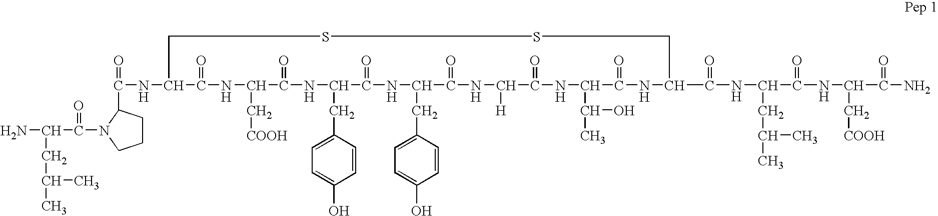
using the double coupling procedure with dicyclohexylcarbodiimide (DIC)/HOBt (1-hydroxybenzotriazole) for 1 hour, followed by HATU/HOAt/DIEA (“HATU”=O—(7-azabenotriazol-1-yl)-N,N,N′,N′-tetramethyluronium hexafluorophosphate) coupling for 1/2 hour. The fully protected resin-bound DTPA-peptide was then subjected to partial deprotection/cleavage procedure using TFA cocktail (94% TFA, 2.5% TES, 2.5% water and 1% anisole). The Cys(Acm) was unaffected by the TFA treatment. The linear DTPA-peptide conjugate was purified by C₁₈ or C₄ reverse-phase HPLC using a gradient of ACN(0.1% TFA) and H₂O (0.1% TFA).

The next step was the deprotection of the Acm groups with the concomitant oxidation of the thiols to the disulfide. The S-protected DTPA-peptide conjugate was dissolved in TFA:anisole (19:1) at 0° C. T1(CF₃COO)₃ was added and the solution stirred for 60 min. The progress of the reaction was monitored by HPLC. At the end of the reaction the TFA was evaporated under a vacuum. The combined pale yellow filtrate was then concentrated-on a SAVANT SpeedVac rotary evaporator system to about 2% of the original volume. This concentrated filtrate was poured into excess cold ether and a white solid separated out. The solution was centrifuged and the pellet obtained was further triturated with ether. After centrifugation, the supernatant was discarded and the pellet was dissolved in 5% AcOH and was purified on RP-HPLC using a gradient of ACN(0.1% TFA) and H₂O (0.1% TFA) to a white fluffy solid in overall yield of 4% based on the starting resin substitution level. The integrity of the purified product was confirmed by electrospray mass spectrometry.

Addition of Gd⁺⁺⁺ to the DTPA-peptide was accomplished according to standard procedures, see, e.g., WO 96/23526.

The dissociation constants (K_{d}) against the dried fibrin and a fibrin-derived peptide DD(E) targets were determined for the Gd-DTPA-Pep 1: K_{d} (dried fibrin)=7.4 μM, K_{d} (DD(E))=2.9 μM. These results indicate that attachment of a chelator did not significantly affect the binding affinity of the PepI polypeptide for fibrin or fibrin-derived fragements.

Additional Gd-DTPA-Gly-[polypeptide] constrast agents were prepared using the fibrin binding polypeptides Trp-Phe-His-Cys-Pro-Tyr-Asp-Leu-Cys-His-Ile-Leu (SEQ ID NO: 21) and Gln-Trp-Glu-Cys-Pro-Tyr-Gly-Leu-Cys-Trp-Ile-Gln (SEQ ID NO: 22). The dissociation constant (K_{d}) against the fibrin-derived peptide DD(E) target was determined for the Gd-DTPA-Gly[SEQ ID NO: 21] fibrin binding moiety: K_{d} (DD(E))=2.90 μM. The dissociation constants (K_{d}) against the dried fibrin and a fibrin-derived peptide DD(E) targets were determined for the Gd-DTPA-Gly[SEQ ID NO: 22] fibrin binding moiety: K_{d} (dried fibrin)=3.6 μM, K_{d} (DD(E))=3.10 μM.

### EXAMPLE 12

### Measurement of Binding Affinity by Equilibrium Dialysis

A solution of fibrinogen (10 mg/ml) was mixed with the MR labeled peptide of Example 11 designated Gd-DTPA-Pep1, at concentrations of 10, 50 and 100 μM. The fibrinogen/peptide solution was placed on one side of a dialysis membrane in an equilibrium dialysis apparatus. The solution was allowed to equilibrate with an equal volume of buffer on the opposite side of the membrane. After equilibrium was achieved (15 hr) the peptide concentration on each side of the membrane was measured by I.C.P. The dissociation constant, K_{d}, was calculated to be 190 μM, by a standard method (Creighton, T. E., Proteins: Structures and Molecular Properties (W.H. Freeman & Co. 1983) p. 342). The peptide showed a specificity for fibrin over fibrinogen of roughly 70-fold (190 μM vs. 2.7 μM).

### EXAMPLE 13

### Measurement of Peptide Binding Affinity to an in vitro Blood Clot

Freshly drawn whole blood was obtained from a human donor and centrifuged at 4000×g for 15 minutes to remove the hematocrit. The blood plasma (supernatant) was removed and dispensed into ten 1.5 ml tubes, 500 μL/tube. The MR labeled peptide of Example 11, GdDTPA-Pep1, was added to each tube at increasing concentrations (2, 5, 8, 10, 15, 20, 30, 50, 75, 100 μM) along with PBS buffer (50 mM sodium phosphate, 150 mM NaCl, pH 7.4) to bring the total volume to 550 μL. The sample was equilibrated at 37° C. A small aliquot (50 μL) was removed for quantitating the total peptide added (measured by Gd I.C.P. analysis after dilution into 0.1 M HNO₃). Clotting was initiated by the addition of 5 μL 2 M CaCl₂, and the samples are incubated at 37° C. for 1 hour. The tubes were spun down briefly (5 sec) and the clot was removed with a pipet tip. The remaining supernatant (˜200 μL) was diluted in HNO₃ and the peptide concentration (equal to the concentration of unbound or free peptide) was measured by Gd I.C.P. analysis. The difference between the total and free peptide concentrations yields the concentration of bound peptide. Calculation of the fibrin binding affinity using the previously described method (Example 4) indicated that the dissociation constant (K_{d}) for GdDTPA-Pep1 is about 20 μM.

Since fibrinogen is a potential competitor to any fibrin-specific agent, measurement of binding affinity to a fibrin clot in the presence of physiological concentrations of fibrinogen is useful for assessing compound efficacy. To measure the affinity of GdDTPA-Pep1 for a blood clot in the presence of fibrinogen, blood is clotted in the presence of varying peptide concentrations, as described above. A thrombin inhibitor such as PPACK (D-Phe-Pro-Arg-chloromethylketone) is added at a concentration sufficient to abolish thrombin activity (10 μM), and fresh blood plasma (containing fibrinogen) is similarly treated. The thrombin-inhibited plasma is added to the blood clot and the clot is incubated for a time sufficient for the peptide to bind to fibrin and other components to come to equilibrium. Free and bound peptide are separated as above, and the apparent K_{d} for fibrin is determined from a plot of bound vs. free peptide concentrations as in Example 4.

### EXAMPLE 14

### Measurement of T₁ Enhancement

To measure the T₁ of water in the presence of the Gd³⁺-DTPA labeled peptide GdDTPA-Pep1 bound to an in vitro clot, samples within the peptide concentration range of 10-200 μM are prepared as in Example 13, above. The samples are mixed in cuvettes and placed in a Bruker Minispec MR spectrophotometer equipped with a 0.47 T magnet. The T₁ of water is measured by a standard inversion recovery pulse sequence. To control for buffer effects, analogous samples are prepared with a diamagnetic analog of the peptide DTPA-Pep1 coordinated with Indium(III).

### EXAMPLE 15

### In Vivo Thrombus Imaging Experiments

¹¹¹Indium-labeled fibrin binding moiety Leu-Pro-Cys-Asp-Tyr-Tyr-Gly-Thr-Cys-Leu-Asp (SEQ ID NO:9) is used to radioimage a human blood clot formed in a segment of the jugular vein of a rabbit. In preparation of the test agent, ¹¹¹In-labelled InCl₃ is used to prepare ¹¹¹In labeled polypeptide to a dose of 200 μCi/kg rabbit. The preparation involves diluting the appropriate volume of ¹¹¹InCl₃ solution (3-5 μL) with 5 μL of 2 M sodium acetate, followed by 20 μL of 0.02 M acetic acid. To this solution is added 25 μL of a 1.3 mM solution of metal-free polypeptide (SEQ ID NO: 9). A 36 mM cold ¹¹¹In-labeled polypeptide is added to make the concentration of ¹¹¹In-labeled peptide 2 μmol/kg rabbit. This solution is shaken and allowed to stand for 30 minutes.

The radiochemical purity is assessed at this point by taking a 1 μL aliquot and diluting with 100 μL water. The diluted sample is injected into a HPLC fitted with a UV and a gamma ray detector using a reverse phase C-18 column. Under the conditions employed, the labeled peptide elutes at 11 minutes, whereas free InCl₃ elutes with the solvent front. Radiochemical purity is expected to be greater than 99%; there is always less than 1% excess unlabeled polypeptide.

After confirming purity, an appropriate amount of ^{99m}Tc-DTPA (˜30 μL) is added to give a specific activity of 200 μCi/kg rabbit. The final solution is diluted to 1.5 mL with saline. To determine if any transmetallation reactions occur, a solution of ^{99m}Tc-DTPA and excess unlabelled polypeptide (SEQ ID NO:9) in acetate buffer is prepared in the absence of any In(III). HPLC analysis at various time points (up to 8 hrs) confirms that the Tc remains coordinated to DTPA and did not coordinated to the polypeptide.

All animal studies are performed in accordance to IACUC protocol (# 413-91-02). White New-Zealand rabbits (2-3 kg) are anesthetized with a cocktail of 50 mg/kg ketamine, 2.5 mg/kg acepromazine, and 5 mg/kg xylazine. The carotid artery is catheterized for blood sampling (PE160). A 1-2 cm segment of the jugular vein, below the facial bifurcation, is isolated using micro clamps (Roboz RS-7438). A human blood ‘clot’ is formed into the isolated segment. To fabricate the clot, 45 μL of CaCl₂ (0.25M) is added to 25 μL of a thrombin solution (3.75 NIHU). The thrombin solution is reconstituted from human thrombin (Sigma T8885) with 67 μL of water. This solution is aspirated with 150 μL of well-mixed human blood immediately prior to injecting it into the isolated jugular segment (with a 27G needle). The clot is allowed to incubate for 30 min. During the incubation period, the facial or femoral vein is catheterized (PE50) for test agent administration. The clamps are released from the segment after the 30-minute incubation.

Imaging is performed using a Siemens E.CAM imager with medium energy collimators. Two different protocols are used. The first protocol is a single isotope study whereby 1.5 mL of test agent (2-10 μM/kg; 200 μCi/kg) is infused through a facial catheter for a period of 15 minutes. 30 s dynamic images (matrix size of 128×128; acquisition zoom of 1.78) are collected throughout the infusion. This is followed by a collection of 5-minute static images (matrix size of 128×128, acquisition zoom of 1.45) at 30 minute, 45 minute and 60 minute time points. Concurrent blood samples are collected every minute for the initial 15 minute period and subsequently at 30 minute, 45 minute, and 60 minute time points. At 60 minutes, the animals are euthanized with pentobarbital (120 mg/kg) and the heart, liver, kidneys and clot removed.

The second protocol is a dual isotopic study. Prior to collecting the dual isotope images, a possible signal cross-over from one isotope to another is evaluated. This is done by acquiring 5-minute static images of the two vials (each filled with 0.5 mL of 50 μCi of ¹¹¹In or ⁹⁹Tc) which are separated by approximately 30 cm. This protocol is performed by using 1.5 mL of dual labeled test agent (2 μM/kg and 200 μCi/kg per agent, see Test Agent Preparation). The dual labeled test agent is infused through a facial vein catheter for a period of 15 minutes. 30 s dynamic images (matrix size of 128×128; acquisition zoom of 1.78) are collected during and after the infusion, for a total period of 60 minutes.

In an alternative method, 1.5 mL of dual labeled test agent (2 μM/kg and 200 μCi/kg per agent) is infused through femoral vein catheter for a period of 5 minutes. 30 s dynamic images (matrix size of 128×128; acquisition zoom of 1.78) are collected during and after the infusion, for a total period of 60 minutes. In both dual isotopic protocols, concurrent blood samples are collected every minute for the first 15 minutes and at the 20 minute, 30 minute, 45 minute and 60 minute time points. At 60 minutes, the animals are euthanized with an overdose of pentobarbital (120 mg/kg). Representative samples from heart, liver, kidneys, and clot are taken for counting.

In all the experiments image analysis is done using Siemen's ICON data processing program or the NucMed-Image (University of St. Louis, St. Louis) program. Time-activity curves for regions of interest (ROI) are measured from the dynamic images. ROI's are selected by visually determining the areas of interest and then using thresholding procedure provided by the NucMed-Image program. Raw data are filtered using 5 point moving-average-filter. In addition, at 30 minute, 45 minute, and 60 minute time points, ten images are averaged to provide higher signal-to-noise images. Blood and organ samples are counted using an Auto-Gamma Counting System gamma counter (Packard Cobra B5003 Series). For the single labeled samples, an energy window of 100-300 keV is used. For the dual labeled samples, energy windows of 170-500 keV and 128-165 keV are used for ¹¹¹In and ⁹⁹Tc, respectively. Possible signal contamination is determined by measuring the signal from ¹¹¹In sample in the ⁹⁹Tc energy window and vice versa at several time points.

### EXAMPLE 16

### Synthesis of Ultrasound Agents

Albumin microparticles containing perfluoropropane are prepared according to the method described in U.S. Pat. No. 4,957,656 (Molecular Biosystems). The microparticles are treated with chromium as described in WO 98/162257 (Molecular Biosystems). After chromium treatment is complete, stirring is stopped and the microparticles are allowed to rise to the surface of the reaction vessel. The liquid below the microparticles is drawn off and replaced with deionized, deaerated, perfluoropropane-saturated water. The suspension is gently stirred and then allowed to rest until the microparticles float back up to the surface, whereupon the wash solution is removed and replaced with a deaerated, perfluoropropane-saturated solution of TCEP (reducing agent) and iminothiolane (thiolating reagent). After two days of stirring under an atmosphere of perfluoropropane, the microparticles are allowed to float to the surface and the solution is drawn off. The microparticles are kept in a minimum amount of water until the peptide reagent is added.

In a separate vessel, fibrin binding polypeptide Leu-Pro-Cys-Asp-Tyr-Tyr-Gly-Thr-Cys-Leu-Asp (SEQ ID NO:9), designated Pep 1 and illustrated below, and an equimolar amount of a heterobifunctional polyethyleneglycol (PEG) crosslinking reagent (5 kDa, Shearwater Polymers), also illustrated below, are stirred for 1 day in 9:1 water:DMF. 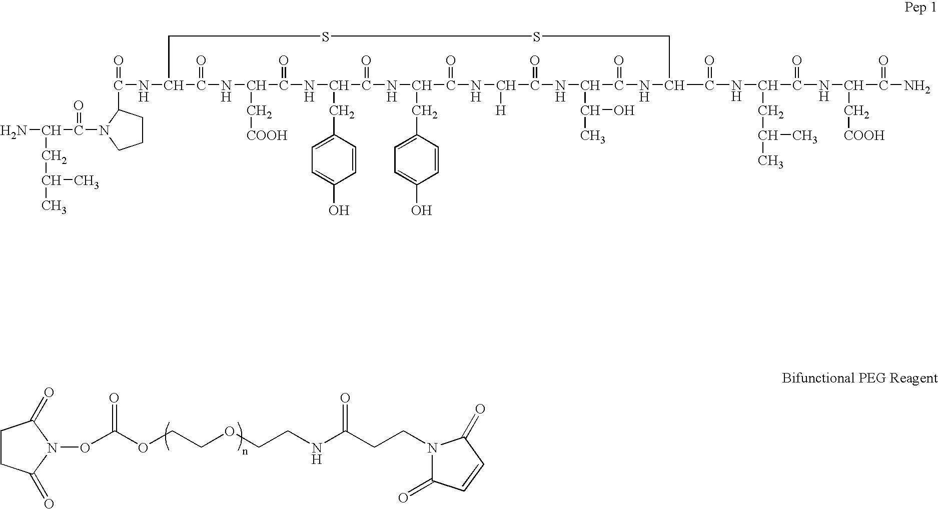
Pep 1 is pegylated at the N terminus by the N-hydroxysuccinimide functional group of the PEG reagent. This solution is rigorously deoxygenated and deaerated by bubbling perfluoropropane and argon through it. The solution of derivatized peptide is buffered to pH˜8 with EPPS, added to the microparticles, and stirred for one day under an atmosphere of perfluoropropane. Exposed thiols on the microparticles are conjugated with the PEG-Pep I construct via the maleimide group of the PEG polymer, to yield fibrin binder-functionalized albumin microparticles encapsulating perfluoropropane gas.

The microparticles are purified by repeated washing with deaerated and deionized water under an atmosphere of perfluoropropane. A solution for injection is prepared by suspending the microparticles in a solution of phosphate-buffered saline so that the solution is of the appropriate pH and tonicity for injection.

### EXAMPLE 17

### Ultrasound Examination of a Blood Clot with Albumin Microparticles

White New-Zealand rabbits (2-3 kg) are anesthetized with a cocktail of 50 mg/kg ketamine, 2.5 mg/kg acepromazine, and 5 mg/kg xylazine. The carotid artery is catheterized for blood sampling. A 1-2 cm segment of the jugular vein, below the facial bifurcation, is isolated using micro clamps. A human blood clot is formed into the isolated segment. To fabricate the clot, 45 μL of CaCl₂ (0.25M) is added to 25 μL of a thrombin solution. The thrombin solution is reconstituted from human thrombin with 67 μL of water. This solution is aspirated with 150 μL of well-mixed human blood immediately prior to injecting it into the isolated jugular segment. The clot is allowed to incubate for 30 min. During the incubation period, the femoral vein is catheterized for test agent administration. The clamps are released from the segment after the incubation.

The test agent is infused through femoral vein catheter over a period of 5 minutes, and 30 minutes after the infusion has terminated, an ultrasound exam is performed with a 5 MHz transducer attached to an HP Sonos 1500 instrument.

### EXAMPLE 18

### Synthesis of Targeted Lipid Microbubbles for Ultrasound

The lipid microbubbles are prepared according to the method described in European patent application EP-A-727 255, wherein fluorinated lipids are conjugated to peptides. An ultrasound agent is made by reacting Pep1 (see illustration above) with (C₁₂F₂₅)(CH₂CH₂O)ₙCO—NHS, isolating the N-terminally derivatized peptide, and subsequently forming the contrast agent. The microbubbles are prepared in the usual manner and an ultrasound examination performed.

### EXAMPLE 19

### Near Infra Red (NIR) Optical Contrast Agent

Pep1 (illustrated above) is stirred in DMF with an equimolar amount of Dye 3 (disclosed in European patent application EP-A-670 374 and illustrated below). The N terminus of the Pep1 polypeptide forms a thiourea link to the dye. After 2 hours, the solution is stripped of solvent by high vacuum, and the conjugated peptide is isolated by preparative reverse phase HPLC. The labeled peptide absorbs and fluoresces in the region 600-1000 nm, an area of the electromagnetic spectrum where body tissues and fluids are largely transparent. A solution of the Pep1-Dye 3 conjugate is injected intravenously and after 30 minutes the patient is examined by measuring absorption of applied light or fluorescence as described by the references cited in WO 96/23524 (Nycomed) and WO 98/48846 (Nycomed).

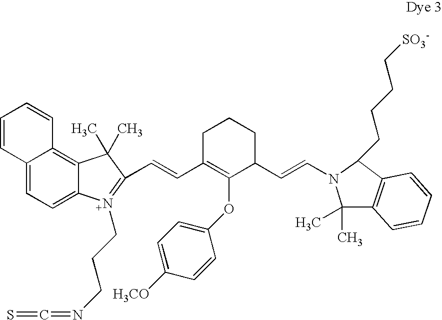

### EXAMPLE 20

### Use of Fibrin Binding Moiety-Fibrin Complexes to Screen Molecular Libraries

Complexes formed between fibrin and fibrin binding moieties of the present invention are used to screen small molecule libraries to discover non-peptidergic, peptidomimetic, or other molecular entities or compounds capable of binding specifically and with high affinity to fibrin. Such novel fibrin binding compounds, which may bind fibrin reversibly or covalently, may find uses comparable or different than those currently envisioned for the fibrin binding moieties of the present invention.

Screening is preferably performed in a high-throughput format. In one version of the screen, fibrin binding moieties are detectably labeled and added in solution to fibrin protein attached to a surface, including, but not limited to, wells of a plastic assay plate (e.g., polystryrene 96-well plate), and allowed to form non-covalent complexes. Fibrin-moiety binding may or may not be allowed to reach equilibrium. Subsequently, a single concentration of unlabeled test compound is added to one well, or multiple concentrations of a test compound is added to multiple wells, and time allowed to enable partial or full equilibrium among fibrin, labeled peptide and test compound. If the test compound binds fibrin, then some labeled peptide is competitively displaced, in proportion to the binding affinity between test compound and fibrin. The well is washed to remove non-fibrin bound labeled peptide and test compound, after which residual non-displaced labeled peptide is detected, such as by an ELISA plate reader. Test compounds capable of displacing a threshold amount of labeled peptide, as adjudged by comparison to appropriate positive and negative controls, are identified as putative fibrin binding molecular entities which are further studied with regard to their physical, chemical, biological, and phannacological properties. In an alternative embodiment of the high-throughput assay, the labeled moiety is mixed with the test compound, each of which is simultaneously added to the well containing the bound fibrin protein. In yet another embodiment, the fibrin binding moiety is attached to the solid surface, and the fibrin is detectably labeled.

Comparison between the effectiveness with which a concentration range of test compound, and an unlabeled version of a fibrin binding peptide of known affinity, displaces a given concentration of labeled peptide of the same species provides an estimate of the fibrin binding affinity of the test compound.

Although a number of embodiments and features have been described above, it will be understood by those skilled in the art that modifications and variations of the described embodiments and features may be made without departing from either the spirit of the invention or the scope of the appended claims. The publications cited herein are incorporated by reference.

1-48. (canceled)

49. An isolated, non-naturally occurring fibrin binding polypeptide comprising:
a polypeptide comprising a cyclic peptide having the amino acid sequence 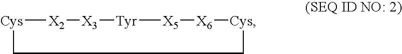
wherein
X₂ is Ala, Glu, Phe, Gly, Ile, Lys, Leu, Met, Arg, Thr, Val, Tyr, Asn, Asp, Gln, His, Ser, or Trp;
X₃ is Ser, Phe, Ala, or Tyr;
X₅ is Gly, Ala or DAla; and
X₆ is Thr, Val or Ser,
wherein said fibrin binding polypeptide has the ability to bind fibrin.

50. The isolated, non-naturally occurring fibrin binding polypeptide according to claim 49, wherein the amino acid residue X₅ is Gly and the amino acid residue X₆ is Thr.

51. The isolated, non-naturally occurring fibrin binding polypeptide according to claim 49, wherein said cyclic peptide comprises the amino acid sequence 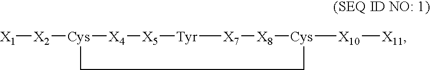
wherein
X₁ is Arg, Asp, His, Leu, or Phe;
X₂ is Ala, Asp, Gly, Pro or Ser;
X₄ is Ala, Glu, Phe, Gly, Ile, Lys, Leu, Met, Arg, Thr, Val, Tyr, Asn, Asp, Gln, His, Ser, or Trp;
X₅ is Ser, Phe, Ala, or Tyr;
X₇ is Gly, Ala or DAla;
X₈ is Thr, Val or Ser;
X₁₀ is His, Leu or Phe; and
X₁₁ is Arg, Asp or His.

52. An isolated, non-naturally occurring fibrin binding polypeptide comprising:
a polypeptide comprising a cyclic peptide having the amino acid sequence 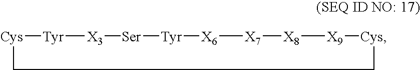
wherein
X₃ is Asn or Asp;
X₆ is Gly or Tyr;
X₇ is His or Val;
X₈ is Pro or Trp; and
X₉ is Trp or Tyr;
wherein said fibrin binding polypeptide has the ability to bind fibrin.

53. The isolated, non-naturally occurring fibrin binding polypeptide according to claim 52, comprising the amino acid sequence 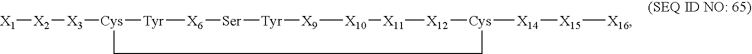
X₁ is Asn or Arg;
X₂ is His or Phe;
X₃ is Gly or Leu;
X₆ is Asn or Asp;
X₉ is Gly or Tyr;
X₁₀ is Val or His;
X₁₁ is Pro or Trp;
X₁₂ is Tyr or Trp;
X₁₄ is Asp or Ser;
X₁₅ is Tyr or His; and
X₁₆ is Ser or His.

54. An isolated, non-naturally occurring fibrin binding polypeptide comprising:
a polypeptide comprising a cyclic peptide having the amino acid sequence 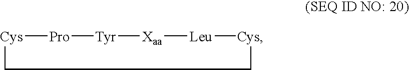
wherein
Xₐₐ is Asp, Gly or Ala, and wherein said fibrin binding polypeptide has the ability to bind fibrin.

55. The isolated, non-naturally occurring fibrin binding polypeptide according to claim 54, wherein said cyclic peptide has the amino acid sequence 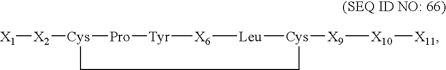
wherein
X₁ is Trp, Phe, His or Tyr;
X₂ is His, Asp or Glu;
X₆ is Asp, Gly or Ala;
X₉ is His, Phe, Tyr or Trp;
X₁₀ is Ile, Leu, or Val; and
X₁₁ is Asn, Gln, Ile, Leu or Val.

56. The isolated, non-naturally occurring fibrin binding polypeptide according to claim 49, said cyclic peptide having an amino acid sequence selected from the group consisting of:

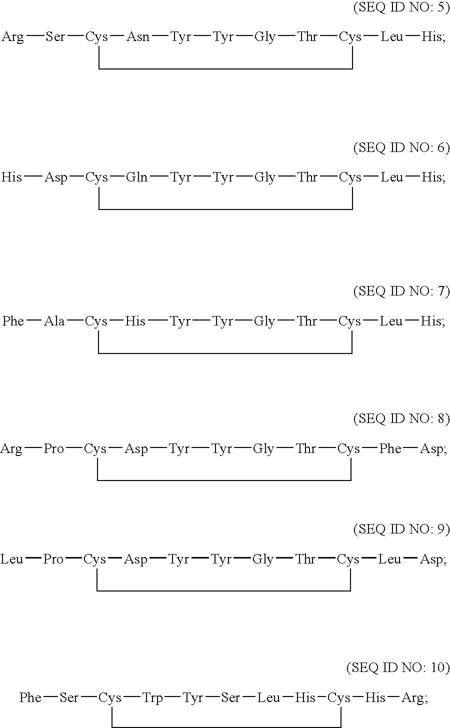

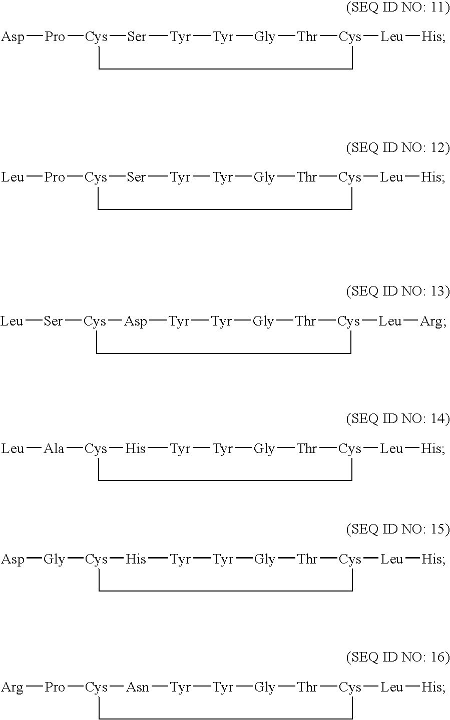

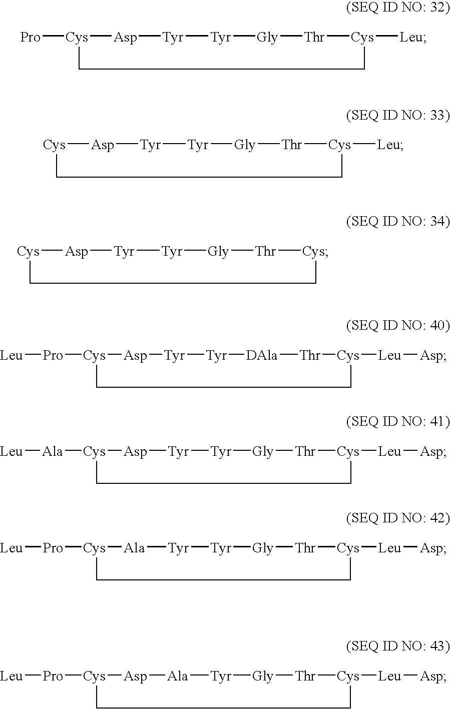

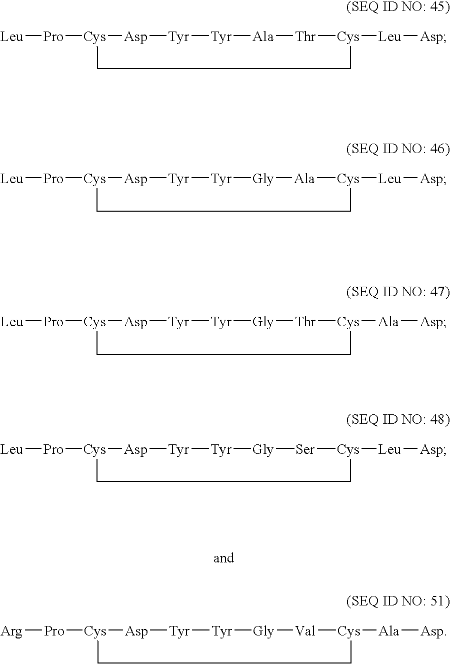

57. The isolated, non-naturally occurring fibrin binding polypeptide according to claim 54, said cyclic peptide having an amino acid sequence selected from the group consisting of:

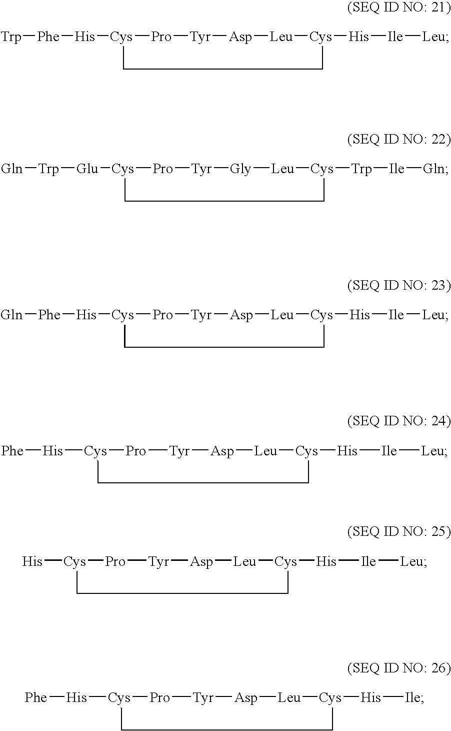

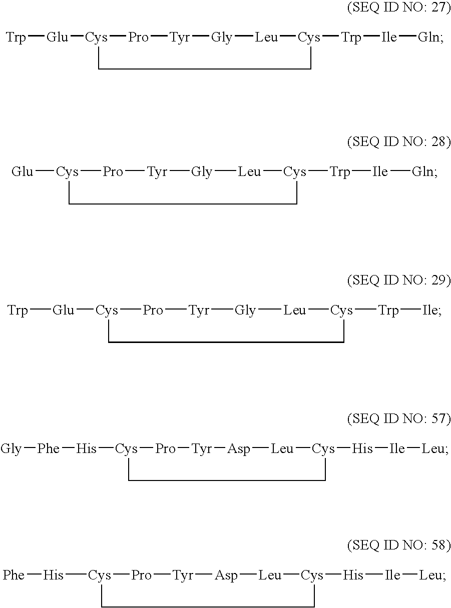

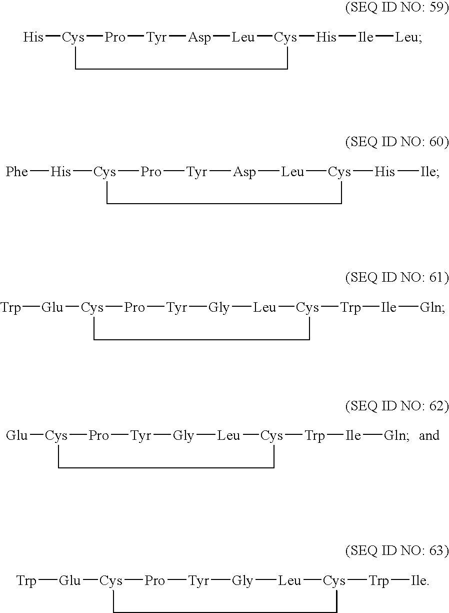

58. The isolated, non-naturally occurring fibrin binding polypeptide according to claim 52, said cyclic peptide having an amino acid sequence selected from the group consisting of:

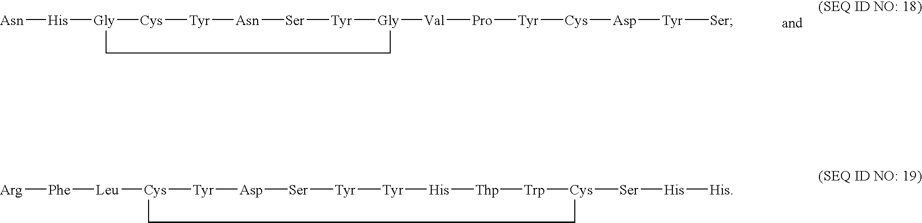

59. An isolated, non-naturally occurring fibrin binding polypeptide according to any one of claims 49 to 58, wherein said polypeptide selectively binds fibrin but not fibrinogen.

60. An isolated, non-naturally occurring fibrin binding polypeptide according to claim 59, wherein said polypeptide has a K_{d} for fibrinogen which is at least 1.5 greater than its K_{d} for fibrin.

61. An isolated, non-naturally occurring fibrin binding polypeptide according to claim 60, wherein said polypeptide has a K_{d} for fibrinogen which is at least 10 greater than its K_{d} for fibrin.

62. An isolated, non-naturally occurring fibrin binding polypeptide according to claim 61, wherein said polypeptide has a K_{d} for fibrinogen which is at least 100 greater than its K_{d} for fibrin.

63. An isolated, non-naturally occurring fibrin binding polypeptide according to claim 62, wherein said polypeptide has a K_{d} for fibrinogen which is at least 1000 greater than its K_{d} for fibrin.

64. A cyclic compound having the ability to bind fibrin and having the formula selected from the group consisting of (I), (II), (III) and (IV): [chemical structure]


SEQUENCE LISTING (69 sequences)

SEQ ID NO: 1 - protein, 11 aa, Artificial Sequence
  synthetic 11-mer microprotein analogues
XXCXXYXXCXX

SEQ ID NO: 2 - protein, 7 aa, Artificial Sequence
  stable binding group of 7 amino acids
CXXYXXC

SEQ ID NO: 3 - protein, 7 aa, Artificial Sequence
  stable binding group of 7 amino acids
CXYYGTC

SEQ ID NO: 4 - protein, 4 aa, Artificial Sequence
  recurrent motif among synthetic fibrin binding peptides
YYGT

SEQ ID NO: 5 - protein, 11 aa, Artificial Sequence
  synthetic fibrin binding peptide
RSCNYYGTCLH

SEQ ID NO: 6 - protein, 11 aa, Artificial Sequence
  synthetic fibrin binding peptide
HDCQYYGTCLH

SEQ ID NO: 7 - protein, 11 aa, Artificial Sequence
  synthetic fibrin binding peptide
FACHYYGTCLH

SEQ ID NO: 8 - protein, 11 aa, Artificial Sequence
  synthetic fibrin binding peptide
RPCDYYGTCFD

SEQ ID NO: 9 - protein, 11 aa, Artificial Sequence
  synthetic fibrin binding peptide
LPCDYYGTCLD

SEQ ID NO: 10 - protein, 11 aa, Artificial Sequence
  synthetic fibrin binding peptide
FSCWYSLHCHR

SEQ ID NO: 11 - protein, 11 aa, Artificial Sequence
  synthetic fibrin binding peptide
DPCSYYGTCLH

SEQ ID NO: 12 - protein, 11 aa, Artificial Sequence
  synthetic fibrin binding peptide
LPCSYYGTCLH

SEQ ID NO: 13 - protein, 11 aa, Artificial Sequence
  synthetic fibrin binding peptide
LSCDYYGTCLR

SEQ ID NO: 14 - protein, 11 aa, Artificial Sequence
  synthetic fibrin binding peptide
LACHYYGTCLH

SEQ ID NO: 15 - protein, 11 aa, Artificial Sequence
  synthetic fibrin binding peptide
DGCHYYGTCLH

SEQ ID NO: 16 - protein, 11 aa, Artificial Sequence
  synthetic fibrin binding peptide
RPCNYYGTCLH

SEQ ID NO: 17 - protein, 10 aa, Artificial Sequence
  synthetic fibrin binding peptide
CYXSYXXXXC

SEQ ID NO: 18 - protein, 16 aa, Artificial Sequence
  synthetic fibrin binding peptide
NHGCYNSYGVPYCDYS

SEQ ID NO: 19 - protein, 16 aa, Artificial Sequence
  synthetic fibrin binding peptide
RFLCYDSYYHWWCSHH

SEQ ID NO: 20 - protein, 6 aa, Artificial Sequence
  stable binding loop of 6 amino acids
CPYXLC

SEQ ID NO: 21 - protein, 12 aa, Artificial Sequence
  synthetic fibrin binding peptide
WFHCPYDLCHIL

SEQ ID NO: 22 - protein, 12 aa, Artificial Sequence
  synthetic fibrin binding peptide
QWECPYGLCWIQ

SEQ ID NO: 23 - protein, 12 aa, Artificial Sequence
  synthetic fibrin binding peptide
GFHCPYDLCHIL

SEQ ID NO: 24 - protein, 11 aa, Artificial Sequence
  synthetic fibrin binding peptide
FHCPYDLCHIL

SEQ ID NO: 25 - protein, 10 aa, Artificial Sequence
  synthetic fibrin binding peptide
HCPYDLCHIL

SEQ ID NO: 26 - protein, 10 aa, Artificial Sequence
  synthetic fibrin binding peptide
FHCPYDLCHI

SEQ ID NO: 27 - protein, 11 aa, Artificial Sequence
  synthetic fibrin binding peptide
WECPYGLCWIQ

SEQ ID NO: 28 - protein, 10 aa, Artificial Sequence
  synthetic fibrin binding peptide
ECPYGLCWIQ

SEQ ID NO: 29 - protein, 10 aa, Artificial Sequence
  synthetic fibrin binding peptide
WECPYGLCWI

SEQ ID NO: 30 - protein, 11 aa, Artificial Sequence
  phage display library template sequence
XXCXXXXXCXX

SEQ ID NO: 31 - protein, 11 aa, Artificial Sequence
  synthetic fibrin binding peptide
XXCXYYGTCXX

SEQ ID NO: 32 - protein, 9 aa, Artificial Sequence
  truncated synthetic fibrin binding peptide
PCDYYGTCL

SEQ ID NO: 33 - protein, 8 aa, Artificial Sequence
  truncated synthetic fibrin binding peptide
CDYYGTCL

SEQ ID NO: 34 - protein, 7 aa, Artificial Sequence
  truncated synthetic fibrin binding peptide
CDYYGTC

SEQ ID NO: 35 - protein, 5 aa, Artificial Sequence
  truncated synthetic fibrin binding peptide
DYYGT

SEQ ID NO: 36 - protein, 16 aa, Artificial Sequence
  variegated template region for phage display library TN10-9
XXXCXXXXXXXXCXXX

SEQ ID NO: 37 - protein, 12 aa, Artificial Sequence
  variegated template region for phage display library TN6-6
XXXCXXXXCXXX

SEQ ID NO: 38 - protein, 11 aa, Artificial Sequence
  linearized fibrin binding peptide
LPSDYYGTSLD

SEQ ID NO: 39 - protein, 11 aa, Artificial Sequence
  linearized fibrin binding peptide
LPXDYYGTXLD

SEQ ID NO: 40 - protein, 11 aa, Artificial Sequence
  substituted fibrin binding peptide
LPCDYYXTCLD

SEQ ID NO: 41 - protein, 11 aa, Artificial Sequence
  alanine-substituted synthetic fibrin binding peptide
LACDYYGTCLD

SEQ ID NO: 42 - protein, 11 aa, Artificial Sequence
  alanine-substituted synthetic fibrin binding peptide
LPCAYYGTCLD

SEQ ID NO: 43 - protein, 11 aa, Artificial Sequence
  alanine-substituted synthetic fibrin binding peptide
LPCDAYGTCLD

SEQ ID NO: 44 - protein, 11 aa, Artificial Sequence
  alanine-substituted synthetic fibrin binding peptide
LPCDYAGTCLD

SEQ ID NO: 45 - protein, 11 aa, Artificial Sequence
  alanine-substituted synthetic fibrin binding peptide
LPCDYYATCLD

SEQ ID NO: 46 - protein, 11 aa, Artificial Sequence
  alanine-substituted synthetic fibrin binding peptide
LPCDYYGACLD

SEQ ID NO: 47 - protein, 11 aa, Artificial Sequence
  alanine-substituted synthetic fibrin binding peptide
LPCDYYGTCAD

SEQ ID NO: 48 - protein, 11 aa, Artificial Sequence
  synthetic fibrin binding peptide
LPCDYYGSCLD

SEQ ID NO: 49 - protein, 11 aa, Artificial Sequence
  synthetic fibrin binding peptide
LPCDYYGXCLD

SEQ ID NO: 50 - protein, 11 aa, Artificial Sequence
  synthetic fibrin binding peptide
LPCDYYGXCLD

SEQ ID NO: 51 - protein, 11 aa, Artificial Sequence
  synthetic fibrin binding peptide
LPCDYYGVCLD

SEQ ID NO: 52 - protein, 11 aa, Artificial Sequence
  synthetic fibrin binding peptide
LPCDYFGTCLD

SEQ ID NO: 53 - protein, 11 aa, Artificial Sequence
  synthetic fibrin binding peptide
LPCDYXGTCLD

SEQ ID NO: 54 - protein, 10 aa, Artificial Sequence
  synthetic fibrin binding peptide
LPCDXGTCLD

SEQ ID NO: 55 - protein, 11 aa, Artificial Sequence
  synthetic fibrin binding peptide
LPCDYXGTCLD

SEQ ID NO: 56 - protein, 11 aa, Artificial Sequence
  synthetic fibrin binding peptide
LPCDYXGTCLD

SEQ ID NO: 57 - protein, 12 aa, Artificial Sequence
  synthetic fibrin binding peptide
GFHCPYDLCHIL

SEQ ID NO: 58 - protein, 11 aa, Artificial Sequence
  synthetic fibrin binding peptide
FHCPYDLCHIL

SEQ ID NO: 59 - protein, 10 aa, Artificial Sequence
  synthetic fibrin binding peptide
HCPYDLCHIL

SEQ ID NO: 60 - protein, 10 aa, Artificial Sequence
  synthetic fibrin binding peptide
FHCPYDLCHI

SEQ ID NO: 61 - protein, 11 aa, Artificial Sequence
  synthetic fibrin binding peptide
WECPYGLCWIQ

SEQ ID NO: 62 - protein, 10 aa, Artificial Sequence
  synthetic fibrin binding peptide
ECPYGLCWIQ

SEQ ID NO: 63 - protein, 10 aa, Artificial Sequence
  synthetic fibrin binding peptide
WECPYGLCWI

SEQ ID NO: 64 - protein, 4 aa, Artificial Sequence
  sequence motif of fibrin binding peptide
YYGX

SEQ ID NO: 65 - protein, 16 aa, Artificial Sequence
  synthetic fibrin binding peptide
XXXCYXSYXXXXCXXX

SEQ ID NO: 66 - protein, 11 aa, Artificial Sequence
  synthetic fibrin binding peptide
XXCPYXLCXXX

SEQ ID NO: 67 - protein, 4 aa, Artificial Sequence
  N-terminus of fibrinogen molecule
GPRV

SEQ ID NO: 68 - protein, 5 aa, Artificial Sequence
  synthetic fibrin binding peptide
DYYGT

SEQ ID NO: 69 - protein, 7 aa, Artificial Sequence
  synthetic fibrin binding peptide
GDYYGTG